(19) United States
(12) Patent Application Publication
(10) Pub. No.: US 2023/0076792 A1
(43) Pub. Date: Mar. 9, 2023

(54) SEQUENTIAL TARGETING IN CROSSLINKING NANO-THERANOSTICS FOR TREATING BRAIN TUMORS

(71) Applicant: The Regents of the University of California, Oakland, CA (US)

(72) Inventors: Yuanpei LI, Oakland, CA (US); Hao WU, Oakland, CA (US); Tzu-yin LIN, Oakland, CA (US)

(21) Appl. No.: 17/785,765

(22) PCT Filed: Dec. 16, 2020
(86) PCT No.: PCT/US2020/065299

§ 371 Date: Jun. 15, 2022

Related U.S. Application Data
(60) Provisional application No. 62/949,284, filed on Dec. 17, 2019.

(51) Int. Cl. A61K 47/69; A61K 47/60; A61K 47/54; A61K 49/08; A61K 49/00

(52) U.S. Cl. CPC A61K 47/6935; A61K 47/60; A61K 47/542; A61K 49/085; A61K 49/0054; A61K 49/0034

(57) ABSTRACT

The present invention provides a compound of Formula (I) as defined herein. The present invention also provides a nanoparticle comprising a plurality of the conjugates of the present invention, and methods of using the nanoparticles for drug delivery, treating a disease, and methods of imaging.

### CROSS-REFERENCES TO RELATED APPLICATIONS

This application claims priority to U.S. Provisional Application No. 62/949,284 filed Dec. 17, 2019, which is incorporated herein in its entirety for all purposes.

### STATEMENT AS TO RIGHTS TO INVENTIONS MADE UNDER FEDERALLY SPONSORED RESEARCH AND DEVELOPMENT

The present invention was made with Government support under Grant No. R01CA199668 awarded by the National Institutes of Health/National Cancer Institute, and Grant No. R01HD086195 awarded by the National Institutes of Health/National Institute of Child Health and Human Development. The Government has certain rights in the invention.

### BACKGROUND

The efficacy of therapeutics for brain tumors is seriously hampered by multiple drug delivery barriers, including severe destabilizing effects in blood circulation, the blood-brain barrier/blood-brain tumor barrier (BBB/BBTB) and limited tumor uptake. Herein is a Sequential Targeting In CrosslinKing (STICK) nano-delivery strategy to circumvent these important physiological barriers to improve drug delivery to brain tumors. STICK nanoparticles (STICK-NPs) could sequentially target BBB/BBTB and brain tumor cells with surface maltobionic acid (MA) and 4-carboxyphenylboronic acid (CBA), respectively, and simultaneously enhance nanoparticle stability with pH-responsive crosslinkages formed by MA and CBA in situ. STICK-NPs exhibited prolonged circulation time (17-fold higher area-under-curve) than free agent, allowing increased opportunities to transpass BBB/BBTB via glucose transporter-mediated transcytosis by MA. Tumor acidic environment then triggered the transformation of STICK-NPs into smaller nanoparticles and revealed secondary CBA targeting moiety for deep tumor penetration and enhanced uptake in tumor cells. STICK-NPs significantly inhibited tumor growth and prolonged the survival time with limited toxicity in mice with aggressive and chemo-resistant diffuse intrinsic pontine glioma. This formulation tackles multiple physiological barriers on-demand with a simple and smart STICK design. Therefore, these features allow STICK-NPs to unleash the potential of brain tumor therapeutics to improve their treatment efficacy.

Patients with aggressive brain tumors, such as glioblastoma (GBM) or pediatric diffuse intrinsic pontine glioma (DIPG), have a dismal prognosis. Particularly, for DIPG, a devastating and aggressive pediatric brain tumor arising in the ventral pons, radiotherapy is currently the only treatment modality. Children with DIPG have only around 2% five-year survival rate. Many chemotherapeutic drugs such as vincristine (VCR) and novel epigenetic modulating agents, such as inhibitors for Histone deacetylase (HDAC), bromodomains of Bromodomain and Extra-terminal motif (BET), and enhancer of zeste homolog 2 (EZH2) showed promising results in the pre-clinical models. Unfortunately, all the clinical trials on the chemotherapy and epigenetic modulating agents failed to improve the treatment outcome compared to radiation alone. The clinical therapeutic effect of these agents is markedly hampered by the poor drug delivery to brain tumors due to several physiological barriers, including strong destabilizing conditions during the circulation in blood (Barrier 1), the blood-brain barrier (BBB)/blood-brain tumor barrier (BBTB) (Barrier 2), poor specificity for targeting tumor cells (Barrier 3) and the relatively weak enhanced permeability and retention effect displayed by brain tumors (FIG. TA). There is a clear and urgent need to develop new therapeutic strategies against brain tumors.

A variety of nanocarriers have been reported attempting to circumvent these biological barriers by actively targeting the receptors or transporters on the BBB/BBTB (e.g. glucose transporter 1 (GLUT1), transferrin receptors, low-density lipoprotein receptor, choline transporter, and amino acids transporters)) and tumor cell/tissue (e.g. sialic acid, integrin family, tropomyosin receptor kinase (TRK) family proteins, epidermal growth factor receptor (EGFR), and folate receptor), respectively. The BBB/BBTB is a highly regulated barrier that controls the traversal of blood-borne substances into the parenchyma of the central nervous system (CNS) and prevents toxic agents, including chemotherapeutic drugs from entering. Several nutrients including glucose are essential for the brain. The transport of glucose into the CNS is facilitated by GLUT1, which is specifically localized on the BBB/BBTB. Several studies have established that GLUT1 as a validated target for transporter-mediated transcytosis of nanoparticles. It is also known that many types of tumor cells (including those of brain tumors) show an increased sialic acid expression on membrane glycoproteins. The hypersialation of a cell membrane during malignant transformation not only contributes to tumor growth and metastasis but also strongly associates with poor prognosis in cancer patients. Thus, targeting tumor cells by their aberrant sialylation has been an attractive strategy for cancer treatment. GLUT1 and sialic acid, had been separately targeted with different nano-carriers, but had never been dually/sequentially targeted with one particle design.

To tackle the challenge in brain tumor delivery, multifunctional nanoparticles must be designed with consideration of the whole-process in drug delivery to brain tumors as well as the dynamic requirements for each delivery stage. Several dual targeting strategies were developed attempting to address the multiple barriers in brain tumor delivery. For example, a dual-targeting peptide angiopep-2 was decorated on the nanoparticles to target both BBB and GBM cells, and this dual-targeting nanocarrier was demonstrated to exhibit superior anti-intracranial GBM effects. Polysorbate 80 (PS 80) was introduced to polymer-bound tratuzumab (anti-Her2 Antibody) to target both BBB and Her2+ breast cancer brain metastasis. In this system, the first step involved in the PS 80-mediated recruitment of circulating apolipoprotein resulting in transcytosis, and the second step was to target Her2 on breast cancer cells with tratuzumab after nanoparticle dissociation. While conceptually attractive, these conventional dual targeting design is usually achieved by simply decorating one or two different targeting moieties on the nanoparticle surface. These moieties ONLY serve for targeting purpose without adding various favorable physical features to the nanoparticle platform to sophisticatedly address the complicated problems in brain tumor delivery.

Herein, is developed a simple-yet-effective Sequential Targeting In CrosslinKing (STICK) nano-delivery approach to improve drug delivery to brain tumors. Strategically, one unique pair of targeting molecules was selected, maltobionic acid (MA, a glucose derivative) and 4-carboxyphenylboronic acid (CBA), as dual targeting moieties for BBB and brain tumor via GLUT1 and sialic acid, respectively, to build interlocking STICK nanoparticles (STICK NPs). Beyond targeting functions, this pair of targeting moieties could form pH-sensitive boronate ester bonds to stabilize the nanocarriers with intermicellar crosslinks, thereby benefiting NP stability in blood circulation (FIG. 1A, Barrier 1). Excess MA (a glucose derivative) on the nanoparticle surface can be recognized by GLUT1 and then trigger the GLUT1-mediated BBB/BBTB transcytosis (FIG. 1A, Barrier 2). Upon exposure to the acidic extracellular pH in solid tumors, the intrinsic MA-CBA boronate ester crosslinkages are cleaved, resulting in the transformation of STICK NPs into small secondary nanoparticles with newly unshielded surface CBA (a synthetic mimic of lectin) which allows deeper tumor penetration and recognition of tumor surface sialic acid, respectively (FIG. 1A, Barrier 3). In this study, is provided a step-by-step proof for the dynamic properties specifically designed to overcome each barrier with STICK approach, including their sequential targeting abilities, pharmacokinetics, and pH-dependent drug release/transformation features. Lastly, it was demonstrated their superior anti-cancer targeting abilities using the dual-modality imaging and anti-cancer efficacies in two different aggressive orthotopic brain tumor models.

### BRIEF SUMMARY OF THE INVENTION

In one embodiment, the present invention provides a compound of Formula I: (R¹)ₘ-D¹-L¹-PEG-L²-D²-(R²)ₙ (I), wherein: each R¹ is independently a peptide, 1,2-dihydroxy compound, or boronic acid derivative; each R² is independently cholic acid or a cholic acid derivative; D¹ and D² are each independently a dendritic polymer having a single focal point group, and a plurality of branched monomer units X; ach branched monomer unit X is a diamino carboxylic acid, a dihydroxy carboxylic acid or a hydroxyl amino carboxylic acid; L¹ and L² are each independently a bond or a linker linked to the focal point group of the dendritic polymer; PEG is a polyethylene glycol (PEG) polymer having a molecular weight of 1-100 kDa; subscript m is an integer from 2 to 8; and subscript n is an integer from 2 to 16.

In another embodiment, the present invention provides a nanoparticle comprising a plurality of first and second conjugates, wherein: each first conjugate is a compound of Formula I wherein each R¹ is independently a peptide, 1,2-dihydroxy compound, sugar compound glucose, or glucose derivative; each second conjugate is a compound of Formula I wherein each R¹ is independently a boronic acid derivative; and the plurality of conjugates self-assemble by forming crosslinking bonds to form a nanoparticle such that the interior of the nanoparticle comprises a hydrophilic interior comprising a plurality of micelles with a hydrophobic core.

In another embodiment, the present invention provides a nanoparticle comprising a hydrophilic exterior and interior, wherein the nanoparticle interior comprises a hydrophilic interior comprising a plurality of micelles having a hydrophobic core and hydrophilic micelle exterior, wherein each micelle comprises a plurality of first and second conjugates, wherein: each first conjugate is a compound of Formula I wherein each R¹ is independently a peptide, 1,2-dihydroxy compound, sugar compound glucose, or glucose derivative; each second conjugate is a compound of Formula I wherein each R¹ is independently a boronic acid derivative; and the plurality of first and second conjugates self-assemble by forming crosslinking bonds to form the micelle with the hydrophobic core, with the crosslinking bonds on the hydrophilic micelle exterior.

In another embodiment, the present invention provides a method of delivering a drug, the method comprising: administering a nanoparticle of the present invention, wherein the nanoparticle further comprises a hydrophilic and/or hydrophobic drug and a plurality of cross-linked bonds; and cleaving the cross-linked bonds in situ, such that the drug is released from the nanoparticle, thereby delivering the drug to a subject in need thereof.

In another embodiment, the present invention provides a method of treating a disease, the method comprising administering a therapeutically effective amount of a nanoparticle of the present invention, wherein the nanoparticle further comprises a hydrophilic and/or hydrophobic drug, to a subject in need thereof.

In another embodiment, the present invention provides a method of imaging, comprising: administering an effective amount of a nanoparticle of the present invention, wherein the nanoparticle further comprises a hydrophilic and/or hydrophobic imaging agent to a subject in need thereof; and imaging the subject.

### BRIEF DESCRIPTION OF THE DRAWINGS

FIG. 1A shows the design of transformable STICK-NPs and detailed multi-barrier tackling mechanisms to brain tumors. The pair of targeting moieties selected to form Sequential Targeting In CrosslinKing (STICK) were maltobionic acid (MA), a glucose derivative, and carboxyphenylboronic acid (CBA), one type of boronic acid, and were built into well-characterized self-assembled micelle formulations (PEG-CA8). STICK-NPs were assembled by a pair of MA4-PEG-CA8 and CBA4-PEG-CA8 with the molar ratio of 9:1 while inter-micelle boronate crosslinkages, STICK, formed between MA and CBA resulting in larger nanoparticle size. Excess MA moieties were on the surface of the nanoparticles, while CBA moieties were firstly shielded inside the STICK to avoid non-specific bindings. Hydrophobic drugs were loaded in the hydrophobic cores of secondary small micelles, while hydrophilic agents were trapped in the hydrophilic space between small micelles. In the following studies several control micelle formulations were used including NM (no targeting), MA-NPs (single BBB targeting), and CBA-NPs (single sialic acid tumor targeting) nanoparticles (inserted table). In detail, STICK-NPs could overcome Barrier 1 (destabilizing condition in the blood) by intermicellar crosslinking strategy, Barrier 2 (BBB/BBTB) by active GLUT1 mediated transcytosis through brain endothelial cells, and Barrier 3 (penetration & tumor cell uptake) by transformation into secondary smaller micelles and reveal of secondary active targeting moiety (CBA) against sialic acid overexpressed on tumor cells in response of acidic extracellular pH in solid tumors. FIG. 1B shows intensity-weighted distribution of MA-NPs, CBA-NPs, NM, and STICK-NPs at pH 7.4 and 6.5. FIG. 1C shows boronate ester bond formation verified by a fluorescence assay based on the indicator of alizarin red S (ARS) (Ex: 468 nm, 0.1 mg/mL). ARS fluorescence decreased along with a dose-dependent increase of MA4-PEG-CA8 concentrations from 0 μM to 40 μM (fixed CBA4-PEG-CA8 with 2.5 μM). This demonstrated the formation of boronate ester bonds between MA4-PEG-CA8 and CBA4-PEG-CA8.FIG. 1D shows Transmission Electron Micrograph (TEM) imaging for visualizing the transformation process of STICK-NPs (92±21 nm) into secondary small micelles (14±3 nm) when changing from pH 7.4 to pH 6.5 at 10 mins (intermediate status) and 24 hours. The size of both large and secondary small micelles measured by TEM were more compatible with the size measured in number-weighted distribution with DLS (pH 7.4: 113.6+45.4 nm and pH 6.5: 14±3 nm, respectively) (FIG. 8F). Of note, the low-contrast nanoparticle outline in the intermediate status represented the empty large nanoparticle with associated secondary small micelles outside. Scale bar, 200 nm or 100 nm (insert). FIG. 1E shows pH-dependent and FIG. 1F shows time-dependent intensity-weighted distribution changes of STICK-NPs under pH 6.5. pH 6.8 appears to be the cut-off value for triggering micelle transformation. FIG. 1G shows the Z-average size of STICK-NPs that was formulated with different solvents (various polarities) and treated with sodium dodecyl sulfate (SDS) or not in PBS. ACN: acetonitrile; DCM: dichloromethane; EtOAc: ethyl acetate.

FIGS. 2A and 2B show cumulative release profile for both hydrophilic (Gd-DTPA) (FIG. 2A) and hydrophobic (Cy7.5) payloads (FIG. 2B) from STICK-NPs and NM in the presence of different pH. A mixture of NM and free Gd was used in (FIG. 2A), as Gd could not be loaded into NM. Drug release study was performed initially at pH 7.4 PBS (grey areas) and was then subjected to pH 6.5 after 4 h (pink areas). Samples were collected at different time points and were measured by inductively coupled plasma mass spectrometry (ICP-MS) for Gd-DTPA level and fluorescence spectrometer for the concentration of Cy7.5. (n=3). FIG. 2C shows in vitro T1-weighted MRI signal of Gd-DTPA, and STICK-NP@Cy@Gd under pH7.4 or pH6.5 at different concentrations acquired by a Bruker Biospec 7T MRI scanner. FIG. 2D shows the Z-average size stability test of STICK-NP@Cy@Gd in the presence of PBS, 10 mg/mL SDS or 10% FBS. (n=3) FIG. 2E show the intensity-weighted distribution changes of STICK-NPs in the presence of different concentrations of glucose (mmol/L). Of note, normal human serum glucose level ranges from 3.9 to 5.5 mmol/L. FIG. 2F show pharmacokinetic profiles of free Cy7.5, STICK-NP@Cy, and NM@Cy (Cy7.5, 10 mg/kg) in jugular vein catheterized rats (n=3). Serum was collected at different time points, and drug concentrations were measured based on fluorescence signals. The error bars were the standard deviation (SD).

FIGS. 3A-3M show multi-barrier tackling mechanism studies for STICK-NPs mediated brain tumor drug delivery process in vitro. FIG. 3A shows diagram for Transwell® (0.4 μm pore size) modeling for Barrier 2 (BBB/BBTB), and the STICK-NP@Cy mediated transcytosis through brain endothelial cells. Mouse brain endothelial cells (bEnd.3) were cultured in the upper chamber. FIG. 3B shows quantitative measurements for the intracellular fluorescence intensity of Cy7.5 in bEnd.3 cells. bEnd.3 cells were incubated with free Cy7.5, STICK-NP@Cy, MA-NP@Cy, CBA-NP@Cy and NM@Cy (Cy7.5: 0.1 mg/mL) and lysed at different time points. To inhibit GLUT1 activity, cells were pre-treated with 40 μM WZB-117 for 1 hour before cellular uptake study in the following (FIGS. 3B-3C). (n=3, **p<0.01, two-way ANOVA). FIG. 3C shows the efficiency of the transcytosis of different formulations with Cy7.5 in the Transwell system as (FIG. 3A). Mouse bEnd.3 cells were seeded in the upper chamber to form a tight junction that was confirmed with >200 Ω·cm² trans-endothelial electrical resistance (TEER). Free Cy7.5, MA-NP@Cy, CBA-NP@Cy, NM@Cy, and STICK-NP@Cy were loaded in the upper chamber and medium in the lower chambers were collected at different time points to measure the fluorescence intensity of Cy7.5. FIG. 3D shows the intensity-weighted distribution of the STICK-NP@Cy presented in the upper chamber, and lower chamber with medium adjusted to pH 7.4 and 6.5, respectively. The size was measured by DLS. n=3. FIG. 3E show representative confocal image of the subcellular distribution of STICK-NP@DiD (red) in the bEnd.3 cells after 1 hour of incubation. Lysotracker (green): lysosome; Hochst 33342 (blue): nuclear staining; Scale bar=20 μm. FIG. 3F show VCR concentrations in normal brain tissue in Balb/c mice with intact BBB at 6 hours post-intravenous injection of STICK-NPs@VCR and other formulations (2 mg/kg). The whole brains were homogenized. VCR was extracted and the concentrations were measured by liquid chromatography-mass spectrometry (LC-MS). FIG. 3G show the diagram depicting barrier 3-tumor uptake and pH-dependent transformation with newly revealed CBA for sialic acid-mediated tumor targeting. FIG. 3H show quantitative fluorescence measurement of total intracellular Cy7.5 with the same treatment at different time points. The Cy7.5 fluorescence intensity was measured through the lysed cells. n=3, **p<0.01, two-way ANOVA. Scale bar=20 μm. Representative quantitative analysis (FIG. 3I) and fluorescence images (FIG. 3J) of U87-MG cellular uptake of free Cy7.5, MA-NP@Cy, CBA-NP@Cy, NM@Cy and STICK-NP@Cy (Cy7.5: 0.1 mg/mL) under different pH (7.4 and 6.5) at 1 hour time point. In one parallel group treated STICK-NPs, the sialic acid expression on the tumor cell surface was augmented with 40 μM azidothymidine (AZT). In another parallel group of treated STICK-NPs, 40 μM free CBA were added to compete with the surface CBA (secondary targeting moiety) on the secondary STICK-NPs. n=3, **p<0.01, two-way ANOVA. FIG. 3K show the diagram of Transwell (0.4 μm pore size) co-culture system with the bEND3 cells in the upper chamber and U87-MG cells in the lower chamber to model Barriers 2+3. Representative fluorescence images (FIG. 3L) and quantitative analysis (FIG. 3M) of U87-MG cells at 1 hour after treatment with free Cy7.5, MA-NP@Cy, CBA-NP@Cy, NM@Cy and STICK-NP@Cy (Cy7.5: 0.1 mg/mL) in the upper chamber. After adding in the upper chamber for one hour, the lower chamber medium was adjusted to pH 7.4 or 6.5 for another hour and the U87-MG cells at lower chamber were incubated for another hour. In a parallel group treated STICK-NPs, GLUT1 activity was pre-inhibited by WZB-117. Scale bar=20 μm. The error bars were the standard deviation (SD).

FIGS. 4A-4D show transforming-dependent tumor penetration study for STICK-NPs. FIG. 4A shows quantitative analysis of the penetration in U87-MG-GFP neurosphere with STICK-NP@DiD (pH 7.4 and 6.5) and other formulations (pH 7.4). The Z-average size of STICK-NP@DiD (pH 7.4) was around 155 nm, while STICK-NP@DiD (pH6.5) and other nanoformulations were around 20 nm. n=3. t-test, **P<0.01. FIG. 4B shows the representative images and quantitative analysis of the penetration of STICK-NP@DiD (red) into DIPG tumor spheroid at 24 hours under pH 7.4 and 6.5. (DiD: 0.05 mg/mL). n=3. t-test, **P<0.01. Scale bar, 100 μm. FIG. 4C shows tissue penetration of STICK-NP@DiD at the normal brain area and implanted DIPG area from the orthotopic mouse model at 16 hours post-injection of STICK-NP@DiD and NM@DiD (Red, 5 mg/kg). DIPG-XIII-P cells were injected into the mouse brainstem to establish the orthotopic model. DIPG bearing mice were injected with STICK-NP@DiD and NM@DiD (Red, 5 mg/kg) for 16 hours. Before sacrificing the mice, Dextran-FITC (green, moleclular weight=70 K) were injected to highlight blood vessels. Penetration distance from the blood vessels was analyzed with Image J (right). DAPI (blue): nuclear staining. Scale bar=100 μm. FIG. 4D shows tissue penetration analysis of STICK@DiD and NM@DiD (Red) beyond the blood vessels (FITC, green) at both normal brain and DIPG tumor sites corresponding to the cross-sections (yellow line) in FIG. 4C.

FIGS. 5A-5F show dual-modality imaging (MRI & NIRF imaging)-guided delivery process of STICK-NPs in orthotopic PDX glioblastoma and PDX DIPG brain tumor models. FIG. 5A shows in vivo T1-weighted MRI and NIRF images (in vivo and ex vivo) on glioblastoma PDX bearing mouse model as indicated time points after iv injections of Cy7.5+Gd, MA-NP@Cy+Gd, CBA-NP@Cy+Gd, NM@Cy+Gd or STICK-NP@Cy@Gd (Gd-DTPA: 25 mg/kg; Cy7.5: 10 mg/kg). Since hydrophilic Gd-DTPA could not be loaded in MA-NP, CBA-NP, NM, free Gd-DTPA was given in conjunction with Cy7.5 loaded nanoparticles as controls. Tumor location was double-verified with T2-weighted MR imaging. FIG. 5B shows quantitative analysis of MRI T1 signal intensity normalized to normal brain tissue. t-test, **p<0.01. FIG. 5C show the NIRF intensity analysis of orthotopic brain tumors based on the whole mouse in vivo imaging at 24 and 48 hours post-injection. n=3, t-test, **p<0.01, *p<0.05. FIG. 5D shows biodistribution analysis based on the Cy7.5 fluorescence intensity (ex vivo NIRF imaging) in PDX GBM bearing mice at 24 hours pos-injections of Cy7.5+Gd, MA-NP@Cy+Gd, CBA-NP@Cy+Gd, NM@Cy+Gd, and STICK-NP@Cy@Gd. n=3, t-test, **p<0.01. FIG. 5E shows representative confocal images from the cryosection of the mouse brain with implanted GBM tumors at 24 hours post-injection of Cy7.5+Gd, MA-NP@Cy+Gd, CBA-NP@Cy+Gd, NM@Cy+Gd, and STICK-NP@Cy@Gd. Blue: DAPI; Green: U87-MG-GFP; Red: Cy7.5. Scale bar=500 μm. The error bars were the standard deviation (SD). FIG. 5F shows T1-weighted MRI and confocal fluorescence imaging, with quantitative analysis, on orthotopic PDX DIPG brain tumor model at 24 hours post-administration of NM@Cy+Gd or STICK-NP@DiD@Gd (Gd-DTPA: 25 mg/kg; DiD: 5 mg/kg as indicated. Before sacrificing the mice, animals were injected with Dextran-FITC(green) to highlight blood vessels. Red: DiD; Scale bar=2 mm.

FIGS. 6A-6E show anti-cancer efficacy studies of STICK-NPs@VCR in the orthotopic PDX DIPG mouse model. FIG. 6A shows tumor progression (blue dotted outline) of orthotopic DIPG mouse model monitored with Gd-enhanced T1-weighted MRI of the same representative mouse from each group on day 0, 6, 12, 18 and 24 day after treatment with PBS, free VCR, NM@VCR, MA-NP@VCR, CBA-NP@VCR, STICK-NP@VCR, Marqibo (VCR 1.5 mg/kg) free VCR2 and STICK-NM@VCR2 (VCR 2 mg/kg) every six days (intravenous injection). Scale bar=10 mm. FIG. 6B shows actual tumor burden was confirmed with histopathology (blue dotted outline) on day 12 post-injection from the same representative mouse with MRI results in FIG. 6A. Scale bar=5 mm. FIG. 6C shows quantitative analysis of the tumor growth curve based on MRI, Kaplan-Meier survival curve is shown in FIG. 6D, and body weight changes is shown in FIG. 6E of the DIPG bearing mice after treatment of STICK-NP, Marqibo, and other formulations. n=6. t-test for tumor burden analysis; Log-rank (Mantel-Cox) test for survival time analysis. **p<0.01, *p<0.05. Of note, all the mice in the treatment groups of PBS, free VCR, NM@VCR, MA-NP@VCR and CBA-NP@VCR died after day 12, while there were survivors in the STICK-NP@VCR groups. Therefore, the tumor growth curve and body weight changes were only plotted based on survived mice in STICK-NP@VCR groups beyond day 12.

FIGS. 7A-7J show characterizations of CBA4-PEG-CA8 and MA4-PEG-CA8 telodendrimers. FIG. 7A shows synthetic process and chemical structure of CBA4-PEG-CA8 and MA4-PEG-CA8 telodendrimers. FIG. 7B shows MALDI-TOF MS and gel permeation chromatography (GPC) of NH2-PEG5k-NH2 polymer, CBA4-PEG-CA8 telodendrimer and MA4-PEG-CA8 telodendrimer. 1H NMR spectra of CBA4-PEG-CA8 in CDCl3 is shown in FIG. 7C and MA4-PEG-CA8 in CDCl3 is shown in FIG. 7D. The chemical shift of PEG chains (3.5-3.7 ppm), cholic acid (0.5-2.4 ppm) and the linked MA (3.2-4.5 ppm) could be observed in the 1HNMR spectra of MA4-PEG-CA8 in CDCl3 by the characteristic peaks. The chemical shift of PEG chains (3.5-3.7 ppm), cholic acid (0.5-2.4 ppm) and the linked CBA (7.2-8.4 ppm) could be observed in the 1HNMR spectra of CBA4-PEG-CA8 in CDCl3 by the characteristic peaks. The effects of the ratio of two telodendrimers on the size is shown in FIG. 7E and PdI in FIG. 7F, (n=3). FIG. 7G shows representative fluorescence images and quantitative expression for the cell uptake of the ratio of two telodendrimers on brain endothelial cell (bEND.3) by loading DiD dye (red). Hoechst (blue): nuclear staining. FIG. 7H shows size distributions (by number weighted) of MA-NPs, CBA-NPs, NM, and STICK-NPs at pH 7.4, and 6.5 pH-dependent in FIG. 7I, and time-dependent in FIG. 7J size changes (by number weighted) of STICK-NPs under pH 6.5. pH 6.8 appears to be the cut-off value for triggering micelle transformation. The error bars were the standard deviation (SD).

FIGS. 8A-8F show characterizations of STICK-NP@Cy@Gd. TEM image of MA-NPs (FIG. 8A) and CBA-NPs (FIG. 8B) micelles are shown. The concentration of the micelles was kept at 1.0 mg/mL. FIG. 8C shows the fluorescence spectrum of STICK-NP@Cy@Gd (Cy7.5: 0.02 mg/mL) in PBS. Ex/Em=820/848 nm. Relaxation rates (r1) for STICK-NP@Cy@Gd at pH 7.4 is shown in FIG. 8D, and pH 6.5 is shown in FIG. 8E. FIG. 8F shows intensity-(left panel) and number- (right panel) weighted distribution of STICK-NP under pH 7.4 (upper panel) and 6.5 (lower panel). Summary table of nanoparticle size measured with different methods. Number-weighted distribution emphasized more on smaller nanoparticles and are usually more compatible with the finding in TEM or Cryo-EM. The slight SIZE difference between TEM and peak mean+/−SD in the number-weighted distribution is because TEM measured the dried-down size, while DLS measured hydrodynamic size.

FIG. 9 shows WZB-117 (GLUT1 inhibitor, 40 μM) restrain brain endothelial cell surface expression of GLUT1. Immunofluorescence localization (a) and quantitative expression (b) of GLUT1 in brain endothelial cell (bEND.3) with WZB-117 (positive control: no treat; negative control: without GLUT1 antibody). c) Quantitative analysis of BBB penetrating efficiency for different VCR formulations after 1-hour incubation in the Transwell (0.4 μm pore size) BBB model system with the bEND.3 cells seeded in the upper chamber. The error bars were the standard deviation (SD).

FIG. 10 shows the efficiency of BBB/BBTB transverse for STICK-NPs. Brain endothelial cell (bEND.3) uptake of free Cy, MA-NP@Cy, CBA-NP@Cy, NM@Cy and STICK-NP@Cy, observed by confocal microscope and quantitative fluorescence intensity. In an additional group, bEND.3 cells were pretreated with WZB-117 (GLUT1 inhibitor) followed by incubation with STICK-NP@Cy. Scale bar=40 μm.

FIG. 11 shows the representative images for the penetration of STICK-NP@DiD (red) into U87-MG-GFP (green) tumor spheroid at 24 h under pH 7.4 and 6.5. (DiD, 0.05 mg/mL). Scale bar=100 μm. White dot line: depth of maximum penetration

FIGS. 12A-12D show dual-model imaging-guided drug delivery of orthotopic GBM(PDX) brain tumor-bearing mice for STICK-NPs. FIG. 12A shows in vivo whole-brain MR imaging of orthotopic PDX brain tumor-bearing mice received Cy+Gd, NM@Cy+Gd, MA-NP@Cy+Gd, CBA-NP@Cy+Gd and STICK-NP@Cy@Gd (Cy7.5: 10 mg/kg, Gd-DTPA: 25 mg/kg) at different time points post-injection. In vivo (FIG. 12B) and ex vivo (FIG. 12C) NIR fluorescence imaging of orthotopic PDX brain tumor bearing mice received Cy+Gd, NM@Cy+Gd, MA-NP@Cy+Gd, CBA-NP@Cy+Gd and STICK-NP@Cy@Gd (Cy7.5: 10 mg/kg, Gd-DTPA: 25/kg) at different time points post-injection are shown. The ex vivo imaging was at 24-hour time point. FIG. 12D show magnified representative confocal images from the cryo-section of the mouse brain with PDX tumour at 24 h post-injection of STICK-NP@Cy@Gd, focused on tumour area. Blue: DAPI; Green: U87-MG-GFP; Red: Cy7.5. Scale bar=500 μm.

FIG. 13 shows tumor growth data plotted of PBS, free VCR, NM@VCR, MA-NP@VCR, CBA-NP@VCR, STICK-NP@VCR, Marqibo (VCR 1.5 mg/kg) free VCR2 and STICK-NP@VCR2 (VCR 2 mg/kg) groups based on MRI.

FIG. 14 shows body weight changes data plotted of PBS, free VCR, NM@VCR, MA-NP@VCR, CBA-NP@VCR, STICK-NP@VCR, Marqibo (VCR 1.5 mg/kg) free VCR2 and STICK-NP@VCR2 (VCR 2 mg/kg) groups.

FIG. 15A shows MR imaging for monitoring of the orthotopic U87-MG tumor (red arrows) burden on day 0, 6, 12 and 18 after treatment with PBS, free VCR, NM@VCR, MA-NP@VCR, CBA-NP@VCR, and STICK-NP@VCR (VCR 2 mg/kg). Scale bar=10 mm. FIG. 15B shows quantitative analysis of the tumor growth curve based on MRI. n=4, t-test, **p<0.01. FIG. 15C shows Kaplan-Meier plots for the survival of orthotopic U87-MG bearing mice treated as (FIG. 15G). (n=4). Log-rank (Mantel-Cox) test, *p<0.05. FIG. 15D shows histopathologic evaluation of the brain/U87-MG brain tumor (black arrows) section on day 12 post-injection. Scale bar=5 mm. The error bars were the standard deviation (SD). FIG. 15E shows body weight changes of U87-MG orthotopic brain tumor mice treated with PBS, VCR, NM@VCR, MA-NP@VCR, CBA-NP@VCR and STICK-NP@VCR on day 1 and 12 as indicated (VCR: 2 mg/kg). (n=4). FIG. 15F shows histopathological evaluation of major organs from the orthotopic U87-MG brain tumor-bearing mice treated with PBS, VCR, NM@VCR, MA-NP@VCR, CBA-NP@VCR and STICK-NP@VCR (VCR: 2 mg/kg) at 12 days post initial treatment (Scale bar=200 μm, H&E stain). The error bars were the standard deviation (SD).

### DETAILED DESCRIPTION OF THE INVENTION

### I. General

The present invention provides a dendrimer compound wherein one end comprises cholic acid or a derivative thereof, and the other end comprises a peptide, 1,2-dihydroxy compound, or boronic acid derivative, which can form nanocarriers by crosslinking. The nanocarriers comprise a plurality of at least two different conjugates which can crosslink, and can comprise hydrophilic and hydrophobic drugs in the interior. The nanocarriers can be used for drug delivery, treating diseases, and imaging.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Peptide” refers to a compound comprising two or more amino acids covalently linked by peptide bonds. As used herein, the term includes amino acid chains of any length, including full-length proteins.

“1,2-dihydroxy compound” refers to a compound that has at least 2 hydroxyl groups which are on adjacent carbon atoms. 1,2-dihydroxy compounds include, but are not limited to sugars, glucose, glucose derivatives, cellulose, oligosaccharide, cyclodextrin, maltobionic acid, glucosamine, sucrose, trehalose, and cellobiose.

“Boronic acid derivative” refers to compound which have a —B(OH)₂ functional group. Examples of boronic acid derivatives include, but are not limited to 3-carboxy-5-nitrophenylboronic acid, 4-carboxyphenylboronic acid, 3-carboxyphenylboronic acid, 2-carboxyphenylboronic acid, 4-(hydroxymethyl)phenylboronic acid, 5-bromo-3-carboxyphenylboronic acid, 2-chloro-4-carboxyphenylboronic acid, 2-chloro-5-carboxyphenylboronic acid, 2-methoxy-5-carboxyphenylboronic acid, 2-carboxy-5-pyridineboronic acid, 6-carboxy-2-fluoropyridine-3-boronic acid, 5-carboxy-2-fluoropyridine-3-boronic acid, 4-carboxy-3-fluorophenylboronic acid, and 4-(bromomethyl)phenylboronic acid.

“Cholic acid” refers to (R)-4-((3R,5S,7R,8R,9S,10S,12S,13R,14S,17R)-3,7,12-trihydroxy-10,13-dimethylhexadecahydro-1H-cyclopenta[a]phenanthren-17-yl)pentanoic acid. Cholic acid is also known as 3α,7α,12α-trihydroxy-5β-cholanoic acid; 3-α,7-α,12-α-Trihydroxy-5-cholan-24-oic acid; 17-β-(1-methyl-3-carboxypropyl)etiocholane-3α,7α,12α-triol; cholalic acid; and cholalin. Cholic acid derivatives and analogs, such as but not limited to, allocholic acid, pythocholic acid, avicholic acid, deoxycholic acid, chenodeoxycholic acid, are also useful in the present invention. Cholic acid derivatives can be designed to modulate the properties of the nanocarriers resulting from telodendrimer assembly, such as micelle stability and membrane activity. For example, the cholic acid derivatives can have hydrophilic faces that are modified with one or more glycerol groups, aminopropanediol groups, or other groups.

“Monomer” and “monomer unit” refer to a diamino carboxylic acid, a dihydroxy carboxylic acid or a hydroxyl amino carboxylic acid. Examples of diamino carboxylic acid groups of the present invention include, but are not limited to, 2,3-diamino propanoic acid, 2,4-diaminobutanoic acid, 2,5-diaminopentanoic acid (ornithine), 2,6-diaminohexanoic acid (lysine), (2-Aminoethyl)-cysteine, 3-amino-2- aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups of the present invention include, but are not limited to, glyceric acid, 2,4-dihydroxybutyric acid, glyceric acid, 2,4-dihydroxybutyric acid, 2,2-Bis(hydroxymethyl)propionic acid and 2,2-Bis(hydroxymethyl)butyric acid. Examples of hydroxyl amino carboxylic acids include, but are not limited to, serine and homoserine. One of skill in the art will appreciate that other monomer units are useful in the present invention.

“Diamino carboxylic acid” refers to a compound which comprises two amine functional groups and at least one carboxyl functional group.

“Dihydroxy carboxylic acid” refers to a compound which comprises two hydroxyl functional groups and at least one carboxyl functional group.

“Hydroxyl amino carboxylic acid” refers to a compound which comprises at least one hydroxyl functional group, at least one amine functional group, and

“Nanoparticle” or “nanocarrier” refers to a particle or carrier resulting from aggregation of the micelles of the present invention. The nanoparticle or nanocarrier can be spherical in shape with a diameter ranging from 1 to 500 nanometers or more. The nanocarrier of the present invention has a hydrophilic interior comprising micelles and a hydrophilic exterior.

“Micelle” refers to an aggregate of compounds of the present invention. The micelles of the present invention has a hydrophobic core and a hydrophilic exterior, which is part of the nanoparticle interior environment.

“Drug” refers to an agent capable of treating and/or ameliorating a condition or disease. A drug may be a hydrophobic drug, which is any drug that repels water, or a hydrophilic drug, which can dissolve in water. Hydrophobic drugs useful in the present invention include, but are not limited to, deoxycholic acid, taxanes, doxorubicin, etoposide, irinotecan, paclitaxel (PTX), docetaxel, Patupilone (epothelone class), rapamycin and platinum drugs. Hydrophilic drugs useful in the present invention include, but are not limited to, gemicitabine, doxorubicin hydrochloride (DOX-HCl), and cyclophosphamide. Other drugs includes non-steroidal anti-inflammatory drugs, and vinca alkaloids such as vinblastine and vincristine. The drugs of the present invention also include prodrug forms. One of skill in the art will appreciate that other drugs are useful in the present invention.

“Imaging” refers to using a device outside of the subject to determine the location of an imaging agent, such as a compound of the present invention. Examples of imaging tools include, but are not limited to, fluorescence microscopy, positron emission tomography (PET), magnetic resonance imaging (MRI), ultrasound, single photon emission computed tomography (SPECT) and x-ray computed tomography (CT).

“Imaging agents” refer to a compound which increases the contrast of structure within the location of the cell or body for imaging methods including, but not limited to fluorescence microscopy, MRI, PET, SPECT, and CT. Imaging agents can emit radiation, fluorescence, magnetic fields or radiowaves. Imaging agents include, but are not limited to radiometal chelators, radiometal atoms or ions, and fluorophores.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Disease” refers to an abnormal condition that negatively affects the structure or function of part or all of an organism, which is not due to any external injury. Diseases are often construed as medical conditions that are associated with specific symptoms and signs. Diseases may include cancer, immunodeficiency, hypersensitivity, allergies, and autoimmune disorders.

### III. Compounds

In some embodiments, the present invention provides a compound of Formula I: (R¹)ₘ-D¹-L¹-PEG-L²-D²-(R²)ₙ (I), wherein: each R¹ is independently a peptide, 1,2-dihydroxy compound, or boronic acid derivative; each R² is independently cholic acid or a cholic acid derivative; D¹ and D² are each independently a dendritic polymer having a single focal point group, and a plurality of branched monomer units X; ach branched monomer unit X is a diamino carboxylic acid, a dihydroxy carboxylic acid or a hydroxyl amino carboxylic acid; L¹ and L² are each independently a bond or a linker linked to the focal point group of the dendritic polymer; PEG is a polyethylene glycol (PEG) polymer having a molecular weight of 1-100 kDa; subscript m is an integer from 2 to 8; and subscript n is an integer from 2 to 16.

Each R¹ of the present invention can include any suitable peptide, 1,2-dihydroxy compound, or boronic acid derivative known by one of skill in the art.

In some embodiments, each R¹ is a peptide. In some embodiments, the peptide is an oligopeptide, cyclic peptide, dipeptide, tripeptide, or tetrapeptide. In some embodiments, the peptide is an oligopeptide such as angiopep-2, lixisenatide, plecanatide, parsabiv, teriparatide, or abaloparatide. In some embodiments, the peptide is angiopep-2.

In some embodiments, each R¹ is a 1,2-dihydroxy compound. In some embodiments, the 1,2-dihydroxy compound is levodopa, dopamine, cellulose, oligosaccharide, cyclodextrin, maltobionic acid, glucosamine, allose, glucose, mannose, galactose, fructose, sucrose, trehalose, or cellobiose. In some embodiments, the 1,2-dihydroxy compound is levodopa, cellulose, oligosaccharide, cyclodextrin, maltobionic acid, glucosamine, sucrose, trehalose, or cellobiose. In some embodiments, the 1,2-dihydroxy compound is maltobionic acid.

In some embodiments, each R¹ is independently a peptide, 1,2-dihydroxy compound, sugar compound, glucose, or glucose derivative. In some embodiments, each R¹ is independently angiopep-2, levodopa, cellulose, oligosaccharide, cyclodextrin, maltobionic acid, glucosamine, sucrose, trehalose, or cellobiose. In some embodiments, each R¹ is independently maltobionic acid.

In some embodiments, each R¹ is independently a boronic acid derivative. In some embodiments, the boronic acid derivative is phenylboronic acid, 2-thienylboronic acid, methylboronic acid, cis-propenylboronic acid, trans-propenylboronic acid, 3-carboxy-5-nitrophenylboronic acid, 4-carboxyphenylboronic acid, 3-carboxyphenylboronic acid, 2-carboxyphenylboronic acid, 4-(hydroxymethyl)phenylboronic acid, 5-bromo-3-carboxyphenylboronic acid, 2-chloro-4-carboxyphenylboronic acid, 2-chloro-5-carboxyphenylboronic acid, 2-methoxy-5-carboxyphenylboronic acid, 2-carboxy-5-pyridineboronic acid, 6-carboxy-2-fluoropyridine-3-boronic acid, 5-carboxy-2-fluoropyridine-3-boronic acid, 4-carboxy-3-fluorophenylboronic acid, or 4-(bromomethyl)phenylboronic acid.

In some embodiments, each R¹ is independently a 3-carboxy-5-nitrophenylboronic acid, 4-carboxyphenylboronic acid, 3-carboxyphenylboronic acid, 2-carboxyphenylboronic acid, 4-(hydroxymethyl)phenylboronic acid, 5-bromo-3-carboxyphenylboronic acid, 2-chloro-4-carboxyphenylboronic acid, 2-chloro-5-carboxyphenylboronic acid, 2-methoxy-5-carboxyphenylboronic acid, 2-carboxy-5-pyridineboronic acid, 6-carboxy-2-fluoropyridine-3-boronic acid, 5-carboxy-2-fluoropyridine-3-boronic acid, 4-carboxy-3-fluorophenylboronic acid, or 4-(bromomethyl)phenylboronic acid. In some embodiments, each R¹ is independently 4-carboxyphenylboronic acid.

R² can be any suitable cholic acid or cholic acid derivative as known by one of skill in the art. Cholic acid derivatives and analogs include, but are not limited to, allocholic acid, pythocholic acid, avicholic acid, deoxycholic acid, and chenodeoxycholic acid. Cholic acid derivatives can be designed to modulate the properties of the nanocarriers resulting from telodendrimer assembly, such as micelle stability and membrane activity. For example, the cholic acid derivatives can have hydrophilic faces that are modified with one or more glycerol groups, aminopropanediol groups, or other groups.

In some embodiments, each R² is independently cholic acid, (3α,5β,7α,12α)-7,12-dihydroxy-3-(2,3-dihydroxy-1-propoxy)-cholic acid (CA-4OH), (3α,5β,7α,12α)-7-hydroxy-3,12-di(2,3-dihydroxy-1-propoxy)-cholic acid (CA-5OH), or (3α,5β,7α,12α)-7,12-dihydroxy-3-(3-amino-2-hydroxy-1-propoxy)-cholic acid (CA-3OH-NH₂). In some embodiments, each R² is cholic acid.

In some embodiments, each branched monomer unit X can be a diamino carboxylic acid, a dihydroxy carboxylic acid and a hydroxyl amino carboxylic acid. In some embodiments, X is a diamino carboxylic acid. In some embodiments, each diamino carboxylic acid can be 2,3-diamino propanoic acid, 2,4-diaminobutanoic acid, 2,5-diaminopentanoic acid (ornithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid or 5-amino-2-(3-aminopropyl) pentanoic acid. In some embodiments, each dihydroxy carboxylic acid can be glyceric acid, 2,4-dihydroxybutyric acid, 2,2-Bis(hydroxymethyl)propionic acid, 2,2-Bis(hydroxymethyl)butyric acid, serine or threonine. In some embodiments, each hydroxyl amino carboxylic acid can be serine or homoserine. In some embodiments, the diamino carboxylic acid is an amino acid.

In some embodiments, each X is independently 2,3-diamino propanoic acid, 2,4-diaminobutanoic acid, 2,5-diaminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. In some embodiments, each X is lysine.

L¹ of the present invention is a bond or any suitable linker. In some embodiments, L¹ is a bond. In some embodiments, L¹ is a linker. The linker can be any suitable linker known by one of skill in the art. In some embodiments, the linker is a C₁₋₂₀ alkylene, C₂₋₂₀ alkenylene, C₂₋₂₀ alkynylene, a PEG polymer, or peptide. In some embodiments, the linker is a C₁₋₁₀ alkylene, C₂₋₁₀ alkenylene, C₂₋₁₀ alkynylene, or a PEG polymer.

L² of the present invention is a bond or any suitable linker. In some embodiments, L² is a bond. In some embodiments, L² is a linker. The linker can be any suitable linker known by one of skill in the art. In some embodiments, the linker is a C₁₋₂₀ alkylene, C₂₋₂₀ alkenylene, C₂₋₂₀ alkynylene, a PEG polymer, or peptide. In some embodiments, the linker is a C₁₋₁₀ alkylene, C₂₋₁₀ alkenylene, C₂₋₁₀ alkynylene, or a PEG polymer.

Polyethylene glycol (PEG) polymers of any size and architecture are useful in the present invention. In some embodiments, PEG has a molecular weight of 1-100 kDa. In some embodiments, PEG has a molecular weight of 1-50 kDa. In some embodiments, PEG has a molecular weight of 1-20 kDa. In some embodiments, PEG has a molecular weight of 1-10 kDa. In some embodiments, PEG has a molecular weight of about 10 kDa, about 9 kDa, about 8 kDa, about 7 kDa, about 6 kDa, about 5 kDa, about 4 kDa, about 3 kDa, about 2 kDa, or about 1 kDa. In some embodiments, PEG has a molecular weight of about 5 kDa. One of skill in the art will appreciate that other PEG polymers and other hydrophilic polymers are useful in the present invention. PEG can be any suitable length.

Subscript m and subscript n can be any suitable integer. In some embodiments, subscript m is an integer from 2 to 8. In some embodiments, subscript m is an integer from 3 to 6. In some embodiments, subscript m is 4. In some embodiments, subscript n is an integer from 2 to 16. In some embodiments, subscript n is an integer from 4 to 12. In some embodiments, subscript n is an integer from 6 to 10. In some embodiments, subscript n is 8. In some embodiments, subscript m is 4 and subscript n is 8.

In some embodiments, the compound has the structure of Formula (Ia):

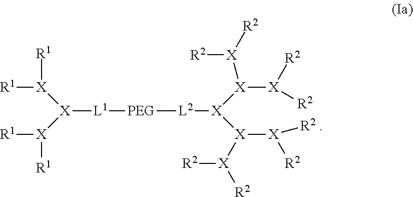

In some embodiments, the compound has the structure of Formula (Ib):

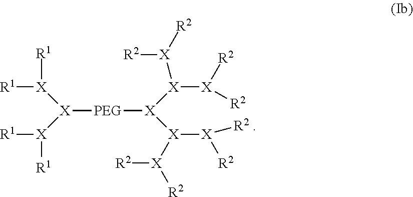

In some embodiments, the present invention provides the compound of Formula (Ib) wherein: each R¹ is maltobionic acid; each R² is cholic acid; each X is lysine; and PEG has a molecular weight of about 5 kDa.

In some embodiments, the present invention provides the compound of Formula (Ib) wherein each R¹ is 4-carboxyphenylboronic acid; each R² is cholic acid; each X is lysine; and PEG has a molecular weight of about 5 kDa.

### IV. Nanoparticles

In some embodiments, the present invention provides a nanoparticle comprising a plurality of first and second conjugates, wherein: each first conjugate is a compound of Formula I wherein each R¹ is independently a peptide, 1,2-dihydroxy compound, sugar compound glucose, or glucose derivative; each second conjugate is a compound of Formula I wherein each R¹ is independently a boronic acid derivative; and the plurality of conjugates self-assemble by forming crosslinking bonds to form a nanoparticle such that the interior of the nanoparticle comprises a hydrophilic interior comprising a plurality of micelles with a hydrophobic core.

In some embodiment, the present invention provides a nanoparticle comprising a hydrophilic exterior and interior, wherein the nanoparticle interior comprises a hydrophilic interior comprising a plurality of micelles having a hydrophobic core and hydrophilic micelle exterior, wherein each micelle comprises a plurality of first and second conjugates, wherein: each first conjugate is a compound of Formula I wherein each R¹ is independently a peptide, 1,2-dihydroxy compound, sugar compound glucose, or glucose derivative; each second conjugate is a compound of Formula I wherein each R¹ is independently a boronic acid derivative; and the plurality of first and second conjugates self-assemble by forming crosslinking bonds to form the micelle with the hydrophobic core, with the crosslinking bonds on the hydrophilic micelle exterior.

The first and second conjugates can be any suitable compound of the present invention. In some embodiments, the first and second conjugate are independently a compound of Formula (Ia). In some embodiments, the first and second conjugates are independently a compound of Formula (Ia) or Formula (Ib). In some embodiments, the first conjugate is a compound of Formula (Ib) wherein R¹ is a peptide, 1,2-dihydroxy compound, sugar compound, glucose, or glucose derivative. In some embodiments, the first conjugate is a compound of Formula (Ib) wherein R¹ is angiopep-2, levodopa, cellulose, oligosaccharide, cyclodextrin, maltobionic acid, glucosamine, sucrose, trehalose, or cellobiose. In some embodiments, the first conjugate is a compound of Formula (Ib) wherein R¹ is maltobionic acid.

In some embodiments, the second conjugate is a compound of Formula (Ib) wherein R¹ is a boronic acid derivative. In some embodiments the second conjugate is a compound of Formula (Ib) wherein R¹ is 3-carboxy-5-nitrophenylboronic acid, 4-carboxyphenylboronic acid, 3-carboxyphenylboronic acid, 2-carboxyphenylboronic acid, 4-(hydroxymethyl)phenylboronic acid, 5-bromo-3-carboxyphenylboronic acid, 2-chloro-4-carboxyphenylboronic acid, 2-chloro-5-carboxyphenylboronic acid, 2-methoxy-5-carboxyphenylboronic acid, 2-carboxy-5-pyridineboronic acid, 6-carboxy-2-fluoropyridine-3-boronic acid, 5-carboxy-2-fluoropyridine-3-boronic acid, 4-carboxy-3-fluorophenylboronic acid, or 4-(bromomethyl)phenylboronic acid. In some embodiments, the first conjugate is a compound of Formula (Ib) wherein R¹ is 4-carboxyphenylboronic acid.

In some embodiments, the first conjugate is a compound of Formula (Ib) wherein: each R¹ is maltobionic acid; each R² is cholic acid; each X is lysine; and PEG has a molecular weight of about 5 kDa, and the second conjugate is a compound of Formula (Ib) wherein each R¹ is 4-carboxyphenylboronic acid; each R² is cholic acid; each X is lysine; and PEG has a molecular weight of about 5 kDa.

In some embodiments, the nanoparticle further comprises a hydrophilic drug or imaging agent. In some embodiments, the hydrophilic drug or imaging agent is encapsulated in the hydrophilic nanocarrier interior and the hydrophilic micelle exterior.

Hydrophilic drugs useful in the present invention can be any suitable hydrophilic drug. In some embodiments, the hydrophilic drug is atenolol, penicillin, ampicillin, Lisinopril, vancomycin, cisplatin, gemicitabine, doxorubicin hydrochloride (DOX-HCl), and cyclophosphamide. In some embodiments, the hydrophilic drug is vancomycin, cisplatin, gemicitabine, doxorubicin hydrochloride (DOX-HCl), and cyclophosphamide. In some embodiments, the hydrophilic drug is cisplatin, gemicitabine, doxorubicin hydrochloride (DOX-HCl), and cyclophosphamide.

Hydrophilic imaging agents useful in the present invention can be any suitable hydrophilic imaging agent. In some embodiments, the hydrophilic imaging agent is calcein, Alexa 680, gadopentetic acid (Gd-DTPA), or indocyanine green (ICG). In some embodiments, the hydrophilic imaging agent is calcein, gadopentetic acid (Gd-DTPA), or indocyanine green (ICG). In some embodiments, the hydrophilic imaging agent is gadopentetic acid (Gd-DTPA), or indocyanine green (ICG).

In some embodiments, the hydrophilic drug or imaging agent is gadopentetic acid (Gd-DTPA), indocyanine green (ICG), cisplatin, gemicitabine, doxorubicin hydrochloride (DOX-HCl), or cyclophosphamide.

In some embodiments, the nanoparticle further comprises a hydrophobic drug or imaging agent. In some embodiments, the hydrophobic drug or imaging agent is encapsulated in the hydrophobic core of the micelle interior in the interior of the nanoparticle.

Hydrophobic drugs useful in the present invention can be any suitable hydrophobic drug. In some embodiments, the hydrophobic drug is resiquimod, gardiquimod, imiquimod, doxorubicin (DOX), vincristine (VCR), everolimus, carmustine, lomustine, temozolomide, lenvatinib mesylate, sorafenib tosylate, regorafenib, Irinotecan, paclitaxel (PTX), Docetaxel, BET inhibitors, OTX015, BET-d246, ABBV-075, I-BET151, I-BET 762, HDAC inhibitors, Valproic acid, Vorinostat, Panobinostat, Entinostat, Ricolinostat, AR-42, JMJD3 inhibitors, GSKJ4, EZH2 inhibitors, Tazemetostat, GSK2816126, MC3629, EGFR inhibitors, Gefitinib, erlotinib, Lapatinib, Osimertinib, AZD92291, IDH inhibitors, enasidenib, ivosidernib, Notch inhibitors, RO4929097, CDK4/6 inhibitors, Palbociclib, Ribociclib, Abemaciclib, PI3K/Akt/mTOR inhibitors, Rapamycin, Buparlisib, Curcumin, or Etoposide. In some embodiments, the hydrophobic drug is doxorubicin (DOX), vincristine (VCR), everolimus, carmustine, lomustine, temozolomide, lenvatinib mesylate, sorafenib tosylate, regorafenib, Irinotecan, paclitaxel (PTX), Docetaxel, BET inhibitors, OTX015, BET-d246, ABBV-075, I-BET151, I-BET 762, HDAC inhibitors, Valproic acid, Vorinostat, Panobinostat, Entinostat, Ricolinostat, AR-42, JMJD3 inhibitors, GSKJ4, EZH2 inhibitors, Tazemetostat, GSK2816126, MC3629, EGFR inhibitors, Gefitinib, erlotinib, Lapatinib, Osimertinib, AZD92291, IDH inhibitors, enasidenib, ivosidemib, Notch inhibitors, RO4929097, CDK4/6 inhibitors, Palbociclib, Ribociclib, Abemaciclib, PI3K/Akt/mTOR inhibitors, Rapamycin, Buparlisib, Curcumin, or Etoposide.

Hydrophobic imaging agents useful in the present invention can be any suitable hydrophobic imaging agent. In some embodiments, the hydrophobic imaging agent is cyanine 5.5 (Cy5.5), cyanine 7.5 (Cy7.5), or 1,1′-Dioctadecyl-3,3,3′,3′-tetramethylindodicarbocyanine 4-chlorobenzenesulfonate (DiD). In some embodiments, the hydrophobic imaging agent is cyanine 7.5 (Cy7.5), or 1,1′-Dioctadecyl-3,3,3′,3′-tetramethylindodicarbocyanine 4-chlorobenzenesulfonate (DiD).

In some embodiments, the hydrophobic drug or imaging agent is cyanine 7.5 (Cy7.5), 1,1′-Dioctadecyl-3,3,3′,3′-tetramethylindodicarbocyanine 4-chlorobenzenesulfonate (DiD), doxorubicin (DOX), vincristine (VCR), everolimus, carmustine, lomustine, temozolomide, lenvatinib mesylate, sorafenib tosylate, regorafenib, Irinotecan, paclitaxel (PTX), Docetaxel, BET inhibitors, OTX015, BET-d246, ABBV-075, I-BET151, I-BET 762, HDAC inhibitors, Valproic acid, Vorinostat, Panobinostat, Entinostat, Ricolinostat, AR-42, JMJD3 inhibitors, GSKJ4, EZH2 inhibitors, Tazemetostat, GSK2816126, MC3629, EGFR inhibitors, Gefitinib, erlotinib, Lapatinib, Osimertinib, AZD92291, IDH inhibitors, enasidenib, ivosidernib, Notch inhibitors, RO4929097, CDK4/6 inhibitors, Palbociclib, Ribociclib, Abemaciclib, PI3K/Akt/mTOR inhibitors, Rapamycin, Buparlisib, Curcumin, or Etoposide.

The ratio of the first and second conjugates can be any suitable ratio known by one of skill in the art and is reported as a molar ratio. In some embodiments, the ratio of the first conjugate to the second conjugate is about 100:1 to 1:10. In some embodiments, the ratio of the first conjugate to the second conjugate is about 50:1 to 1:10. In some embodiments, the ratio of the first conjugate to the second conjugate is about 25:1 to 1:10. In some embodiments, the ratio of the first conjugate to the second conjugate is about 10:1 to 1:10. In some embodiments, the ratio of the first conjugate to the second conjugate is about 50:1, 25:1, 10:1, 9:1, 5:1, 1:1, 1:5, or 1:10. In some embodiments, the ratio of the first conjugate to the second conjugate is about 10:1, 9:1, 5:1, 1:1, 1:5, or 1:10. In some embodiments, the ratio of the first conjugate to the second conjugate is about 10:1, 9:1, and 5:1. In some embodiments, the ratio of the first conjugate to the second conjugate is about 9:1.

### V. Pharmaceutical Composition Formulations

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989).

### VI. Administration

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### VI. Methods of Treatment

In some embodiments, the present invention provides a method of delivering a drug, the method comprising: administering a nanoparticle of the present invention, wherein the nanoparticle further comprises a hydrophilic and/or hydrophobic drug and a plurality of cross-linked bonds; and cleaving the cross-linked bonds in situ, such that the drug is released from the nanoparticle, thereby delivering the drug to a subject in need thereof.

The nanoparticle of the present invention can comprise a plurality of cross-linked bonds which can be cleaved in situ under suitable pH conditions such that the drug is released from the nanoparticle. In some embodiments, the pH is 7 or less. In some embodiments, the pH is about 6.5 or less. In some embodiments, the pH is from 1 to 7. In some embodiments, the pH is from 1 to 6.5. In some embodiments, the pH is from 2 to 6.5. In some embodiments, the pH I from 4 to 6.5. In some embodiments, the pH is about 4, 4.5, 5, 5.5, 6, or 6.5. In some embodiments, the pH is about 6.5.

The hydrophobic drugs useful in the present invention can be any hydrophobic drug known by one of skill in the art. Hydrophobic drugs useful in the present invention include, but are not limited to, deoxycholic acid, deoxycholate, resiquimod, gardiquimod, imiquimod, a taxane (e.g., paclitaxel, docetaxel, cabazitaxel, Baccatin III, 10-deacetylbaccatin, Hongdoushan A, Hongdoushan B, or Hongdoushan C), doxorubicin, etoposide, irinotecan, SN-38, cyclosporin A, podophyllotoxin, Carmustine, Amphotericin, Ixabepilone, Patupilone (epothelone class), rapamycin and platinum drugs. Hydrophilic drugs useful in the present invention include, but are not limited to, atenolol, penicillin, ampicillin, Lisinopril, vancomycin, cisplatin, gemicitabine, doxorubicin hydrochloride (DOX-HCl), and cyclophosphamide. Other drugs includes non-steroidal anti-inflammatory drugs, and vinca alkaloids such as vinblastine and vincristine.

Drugs useful in the present invention include chemotherapeutic agents and immunomodulcatory agents. For example, the drugs can be, but are not limited to, deoxycholic acid, or the salt form deoxycholate, pembrolizumab, nivolumab, cemiplimab, a taxane (e.g., paclitaxel, docetaxel, cabazitaxel, Baccatin III, 10-deacetylbaccatin, Hongdoushan A, Hongdoushan B, or Hongdoushan C), doxorubicin, etoposide, irinotecan, SN-38, cyclosporin A, podophyllotoxin, Carmustine, Amphotericin, Ixabepilone, Patupilone (epothelone class), rapamycin and platinum drugs. Other drugs include non-steroidal anti-inflammatory drugs, and vinca alkaloids such as vinblastine and vincristine. In some embodiments, the drug is paclitaxel, resiquimod, gardiquimod, or deoxycholate.

In some embodiments, the hydrophilic and/or hydrophobic drug is doxorubicin hydrochloride (DOX-HCl), doxorubicin (DOX), vincristine (VCR), or paclitaxel (PTX).

In some embodiments, the present invention provides a method of treating a disease, the method comprising administering a therapeutically effective amount of a nanoparticle of the present invention, wherein the nanoparticle further comprises a hydrophilic and/or hydrophobic drug, to a subject in need thereof.

The nanocarriers of the present invention can be administered to a subject for treatment, of diseases including cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 2008) for additional cancers).

Other diseases that can be treated by the nanocarriers of the present invention include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., atherosclerosis, myositis, neurological conditions such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Behcet's syndrome).

In some embodiments, the disease is cancer. In some embodiments, the disease is selected from the group consisting of bladder cancer, brain cancer, brain metastases, breast cancer, cervical cancer, cholangiocarcinoma, colorectal cancer, esophageal cancer, gall bladder cancer, gastric cancer, glioblastoma, diffuse intrinsic pontine glioma, intestinal cancer, head and neck cancer, leukemia, liver cancer, lung cancer, melanoma, myeloma, ovarian cancer, pancreatic cancer and uterine cancer. In some embodiments, the disease is selected from the group consisting of bladder cancer, breast cancer, colorectal cancer, esophageal cancer, glioblastoma, head and neck cancer, leukemia, lung cancer, myeloma, ovarian cancer, and pancreatic cancer.

In some embodiments, the disease is cancer. In some embodiments, the disease is glioblastoma, diffuse intrinsic pontine glioma, brain metastases, lung cancer, breast cancer, colon cancer, kidney, cancer, or melanoma.

The hydrophilic and hydrophobic drugs useful in the present invention are listed above. In some embodiments, the hydrophilic and/or hydrophobic drug is doxorubicin hydrochloride (DOX-HCl), doxorubicin (DOX), vincristine (VCR), or paclitaxel (PTX).

### VIII. Methods of Imaging

In some embodiments, the present invention provides a method of imaging, comprising: administering an effective amount of a nanoparticle of the present invention, wherein the nanoparticle further comprises a hydrophilic and/or hydrophobic imaging agent to a subject in need thereof; and imaging the subject.

The imaging techniques useful in the present invention are any suitable techniques known by one of skill in the art. In some embodiments, the imaging technique is positron emission tomography (PET), magnetic resonance imaging (MRI), ultrasound, single photon emission computed tomography (SPECT), x-ray computed tomography (CT), echocardiography, fluorescence spectroscopy, near-infrared fluorescence (NIRF) spectroscopy, or a combination thereof. In some embodiments, the imaging technique is MRI, fluorescence spectroscopy, NIRF spectroscopy, or a combination thereof. In some embodiments, the imaging technique is MRI, NIRF spectroscopy, or a combination thereof.

The imaging agents useful in the present invention can be any imaging agent known by one of skill in the art. The imaging agents of the present invention can be either hydrophobic or hydrophilic imaging agent. Imaging agents include, but are not limited to, paramagnetic agents, optical probes, and radionuclides. Paramagnetic agents are imaging agents that are magnetic under an externally applied field. Examples of paramagnetic agents include, but are not limited to, iron particles including nanoparticles. Optical probes are fluorescent compounds that can be detected by excitation at one wavelength of radiation and detection at a second, different, wavelength of radiation. Optical probes useful in the present invention include, but are not limited to, indocyanine green (ICG), Cy5.5, Cy7.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo radioactive decay. Radionuclides useful in the present invention include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁹⁰Sr, ⁹⁰Y, ⁹⁹Tc, ^{99m}Tc, ¹¹¹In, ¹²³I, ¹²⁴I, ¹²⁵I, ¹²⁹I, ¹³¹I, ¹³⁷Cs ¹⁷⁷Lu, ¹⁸⁶Re, ¹⁸⁸Re, ²¹¹At, Rn, Ra, Th, U, Pu and ²⁴¹Am.

In some embodiments, the hydrophilic and/or hydrophobic imaging agent is gadopentetic acid (Gd-DTPA), indocyanine green (ICG), cyanine 7.5 (Cy7.5), or 1,1′-Dioctadecyl-3,3,3′,3′-tetramethylindodicarbocyanine 4-chlorobenzenesulfonate (DiD).

### IX. Examples

### Example 1. Synthesis of Telodendrimers

Chemicals. O-(2-Aminoethyl)-O′-[2-(Boc-amino) ethyl] decaethylene glycol (NH2-PEG-Boc, Mw: 5000 Da) and O-(2-Aminoethyl)polyethylene glycol (NH2-PEG, Mw: 5000 Da) were purchased from Rapp Polymere (Germany). 4-carboxyphenylboronic acid (CBA) and maltobionic acid (MA) were obtained from Combi-Blocks (San Diego, Calif.). (Fmoc)lys(Boc)-OH was purchased from AnaSpec Inc (San Jose, Calif.). Gadopentetic acid (Gd-DTPA) was purchased from Alizarin red S (ARS), cyclohexanone, phosphorus(V) oxychloride (POCl3) 1,1,2-trimethylbenz[e]indole 3-iodopropionic acid, sodium dodecyl sulfate (SDS), D-fructose, cholic acid, azidothymidine(AZT) and all other chemicals were purchased from Sigma-Aldrich (St. Louis). CY7.5 dye was synthesized in lab.

Syntheses of PEG-CA8, Boc-NH-PEG-CA8, MA4-PEG-CA8 and CBA4-PEG-CA8 telodendrimers. The PEG5k-CA8 telodendrimer and Boc-NH-PEG-CA8 telodendrimer were synthesized according to previously reported methods to prepare the non-cross-linked micelle (NM) and synthesize the precursor of crosslinked micelle, respectively, by NH2-PEG and NH2-PEG-Boc. MA4-PEG-CA8 and CBA4-PEG-CA8 telodendrimers were synthesized by using Boc-NH-PEG-CA8 as a starting material via solution phase condensation reactions as described previously. Briefly, the Boc groups of Boc-NH-PEG-CA8 were removed by the treatment with 50% (v/v) trifluoroacetic acid in dimethylformamide (DMF) and NH2-PEG-CA8 were precipitated by adding cold ether and then washed with cold ether twice. (Fmoc)Lys(Fmoc)-OH (4 eq.) was coupled onto the N terminus of NH2-PEG-CA8 using DIC and HOBt as coupling reagents until a negative Kaiser test result was obtained, thereby indicating completion of the coupling reaction resulting in (Fmoc)Lys(Fmoc)-PEG-CA8. This polymer was then precipitated by adding cold ether and washed with cold ether twice. Then, Fmoc groups were removed by treating the polymer with 20% (v/v) 4-methylpiperidine in dimethylformamide (DMF), followed by precipitation and washing steps as described above. White powder precipitate was dried under vacuum and two couplings of (Fmoc)Lys(Fmoc)-OH were carried out respectively to generate the second generation of dendritic polylysine terminated with four Fmoc groups on one end of PEG-CA8. MA and CBA were coupled to the terminal end of dendritic polylysine after Fmoc removal, resulting in MA4-PEG-CA8 telodendrimer and CBA4-PEG-CA8 telodendrimer, respectively. The two telodendrimers were then dialyzed and finally lyophilized.

The mass spectra of the telodendrimers were collected on the ABI 4700 MALDI-TOF/TOF mass spectrometer (linear mode), using 2,5-dihydroxybenzoic acid as a matrix. The molecular weight distribution and polydispersity index (PdI) were collected by the gel permeation chromatography (GPC, Waters e2695, mobile phase 0.1 M NH4Ac aqueous solution). 1H-NMR spectra of the polymers were recorded on a Bruker 800 MHz Avance Nuclear Magnetic Resonance Spectrometer using CDCl₃ as solvents.

### Example 2. Nanoparticles

Preparation of nanoparticles. MA4-PEG-CA8 and CBA4-PEG-CA8 (different ratio) were first dissolved in certain polar solvent, e.g. chloroform, in a round bottom flask. The solvent was evaporated under vacuum to form a thin film. PBS buffer was added to re-hydrate the thin film, followed by 30 min of sonication. Boronate ester bonds formed between CBA and MA of adjacent telodendrimers, upon self-assembly in PBS, resulted in the formation of cross-linked STICK-NPs. The nanoparticle solution was filtered with 0.22 μm to sterilize the sample. Similarly, NM micelles, MA-NPs micelles and CBA-NPs micelles were prepared by using 10 mg PEG-CA8, 9 mg MA4-PEG-CA8 and 1 mg PEG-CA8, and 1 mg CBA4-PEG-CA8 and 9 mg PEG-CA8, in 1 mL PBS, respectively. No crosslinks were formed in those three control micelles.

Characterizations of nanoparticles. The size and size distribution of the nanoparticles were measured by dynamic light scattering (DLS) instruments (Malvern, Nano-ZS). The telodendrimer concentrations of the nanoparticles were kept at 1.0 mg/mL for DLS measurements. Each sample was measured three times with an acquisition time at room temperature. The data were analyzed by Malvern Zetasizer Software and values were reported as the means for each triplicate measurement. The morphology of nanoparticles was observed on a TALOS L120C TEM transmission electron microscope (TEM) at pH 7.4 and 6.5 (at 10 min and 24 h). The aqueous nanoparticle solution (1.0 mg/mL) was deposited onto copper grids and measured at room temperature. 1H-NMR spectra of the telodendrimers were recorded using a Bruker 800 MHz spectrometer in CDCl₃.

Investigation of the formation of STICK-NPs. The MA4-PEG-CA8 (0.9 mg) and CBA4-PEG-CA8 (0.1 mg) were dissolved in 1 mL water, methanol, acetonitrile (ACN), dichloromethane (DCM), ethyl acetate and methylbenzene, respectively, and the size of these nanoparticles was tested by DLS. Then, the solvent was evaporated under vacuum to form a thin film. PBS buffer (1 mL) was added to re-hydrate the thin film, followed by 30 min of sonication. The size and morphology of these nanoparticles were tested by DLS and TEM. In addition, 0.1 mL, 20 mg/mL SDS solution was added to these nanoparticles to test the formation of boronate cross-linkages by DLS.

TABLE 1
The loading rate of hydrophilic and hydrophobic agents by STICK-NPs
(20 mg/mL).

| Co-loading or | | Size | Hydrophobic | Loading | Size |
|---|---|---|---|---|---|
| hydrophilic agent | Loading rate | (DLS) | agent | rate | (DLS) |
| Gd-DTPA (2.5 mg) | 82.4% (by | 146 nm | Cy7.5 (1 mg) | 91% | 172 nm |
| & Cy7.5 (1 mg) | ICP) & 90 % | | DiD (0.5 mg) | 92.6% | 155 nm |
| ICG (1 mg) | 98% | 162 nm | VCR (0.2 mg) | 89.1% | 162 nm |
| DOX HCl (2 mg) | 81.2% | 171 nm | PTX (2 mg) | 87.5% | 149 nm |

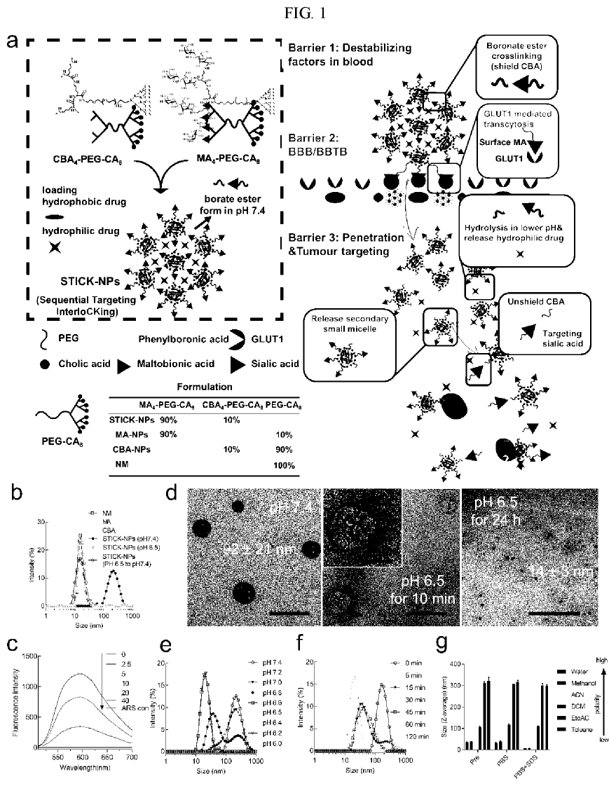

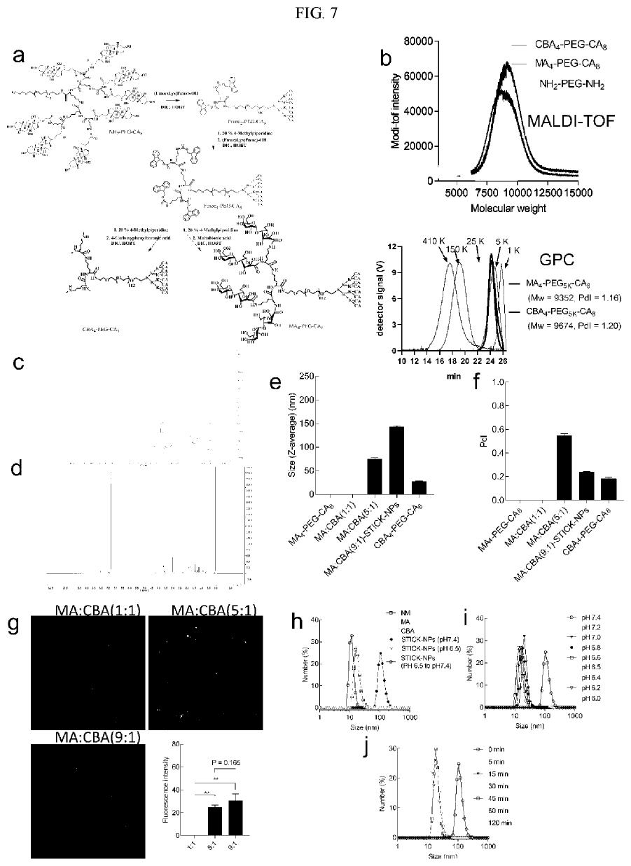

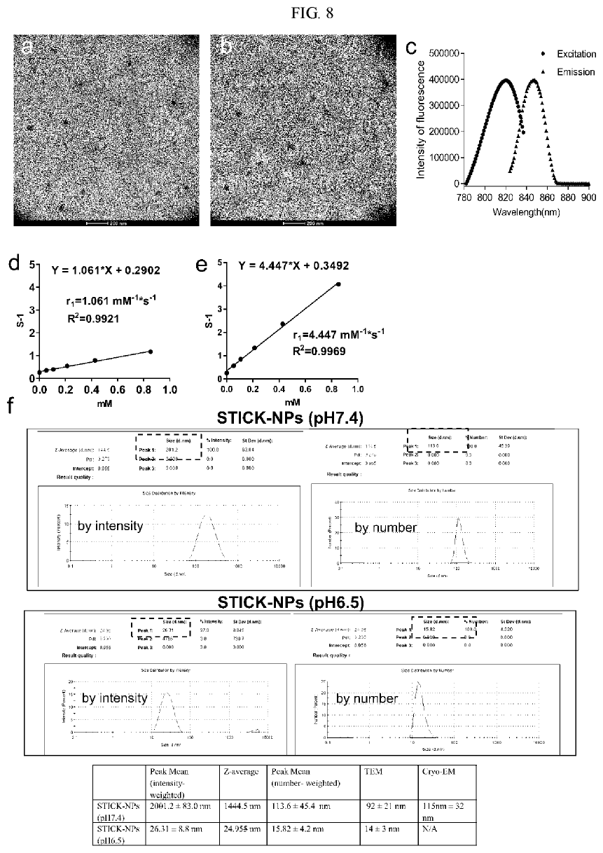

The principle of STICK approach is to select two different targeting moieties which could also form stimuli-responsive crosslinkages. Considering the barrier 2 and 3 in brain tumor delivery, MA was chosen, glucose derivative, for GLUT1-mediated transcytosis through the BBB/BBTB endothelial cells, and CBA which is a type of boronic acid that can target highly expressed sialic acid on brain tumor cells. A pair of the telodendrimers, MA4-PEG-CA8 and CBA4- PEG-CA8, (FIG. 1A; FIG. 7A) were synthesized, and the molecular weight, polydispersity index (PdI) and chemical structure of two telodendrimers were characterized by matrix-assisted laser desorption/ionization time of flight mass spectrometry (MALDI-TOF MS), gel permeation chromatography (GPC) (FIG. 7B) and 1H nuclear magnetic resonance spectroscopy (1H-NMR) (FIG. 7C-7D), respectively. Similar to PEG-CA8, both MA4-PEG-CA8 and CBA4-PEG-CA8 telodendrimers could individually form well-defined small (Z-average size: ˜24 nm) spherical nanoparticles with a narrow size distribution (FIG. 1B; FIGS. 7E-7F, and FIG. 8A-8B). In order to realize sequential targeting, for the first stage brain endothelial cells, a higher ratio of MA telodendrimer is required to remain free MA targeting moiety on the nanoparticle surface after forming boronate ester bonds with a lower ratio of CBA telodendrimer (FIG. 1C). Thus, different ratios (1:1, 5:1, and 9:1) of MA4-PEG-CA8 and CBA4-PEG-CA8 were mixed to form STICK-NPs. The intensity-weighted distribution, polydispersity index (PdI), and brain endothelial cell targeting ability were assessed using dynamic light scatter (DLS) and fluorescence image, respectively (FIG. 7E-7G). It was discovered that with the increase of the MA4-PEG-CA8 ratio, the size of resulting nanoparticles and endothelial cell targeting ability increased, the nanoparticle PdI decreased. Considering all the factors mentioned above, the 9:1 ratio of MA4-PEG-CA8 and CBA4-PEG-CA8 were determined as the optimal ratio as this formulation gave the most uniform nanoparticle (lowest PdI) among all ratios. Other ratios appeared to form both large and small nanoparticles indicating possible increased intramicelle crosslinkages (formed inside small micelles). Unlike the small micelles (around ˜14 nm by TEM) formed based on one species of telodendrimers (FIG. 8A-8B), STICK-NPs were relatively large (Z-average size: 144 nm; TEM size: 92±21 nm), spherical in shape, and contained numerous smaller secondary micelles with a comparable size to non-crosslinked micelles (FIGS. 1B, 1D). With the decrease of the pH (7.4 to 6.5), boronate ester bonds degraded and STICK-NPs were dissociated into numerous smaller secondary micelles (Z-average size: ˜25 nm, FIG. 1B; TEM size: 14±3 nm, FIG. 1D). Of note, Z-average size and intensity-weighted distribution were exclusively used in this study to better describe the process of the transformation. Nevertheless, number-weighted distributions of STICK-NP under both pH 7.4 and 6.5 were also included in the FIG. 8F, to better explain the TEM findings (FIG. 1D). The cut-off pH value for pH-dependent transformation of STICK-NPs is around 6.8 (FIG. 1E), and the transformation took place as early as 5 min and completed at around 1 hour upon exposure to pH 6.5 environment (FIG. 1F).

Another particular feature of STICK-NPs is their capability to encapsulate both hydrophobic and hydrophilic payloads, which offers a significant advantage over conventional micelles that generally only load hydrophobic drugs. STICK-NPs were self-assembled selectively in low-polarity solvents into core-inversible micelles driven by hydrophilic interactions and formed plenty of hydrophilic spaces as reported in another study. The formation of inter-micellar crosslinkages preserves the hydrophilic spaces in the subsequent assembly procedures in aqueous solution together with the newly formed hydrophobic cores. This allows the trapping of hydrophilic agents between secondary micelles and hydrophobic agents in the hydrophobic cholic acid core, like other control micelles (FIG. 1A). It was demonstrated that both hydrophilic agents (e.g. indocyanine green (ICG), gadopentetic acid (Gd-DTPA), doxorubicin hydrochloride (DOX HCl)) and hydrophobic agents (e.g. Cyanine7.5 (Cy7.5), 1,1′-Dioctadecyl-3,3,3′,3′-tetramethylindodicarbocyanine 4-chlorobenzenesulfonate (DiD), VCR and paclitaxel (PTX)) could be encapsulated into STICK-NPs with high loading efficiency (Table 1). Gd-DTPA and Cy7.5 could be co-loaded together into STICK-NPs with a diameter of 146 nm for a variety of theranostic applications as shown in the subsequent sections.

STICK-NPs were formulated in diverse solvents with various polarities (FIG. 1G). In a nonpolar solvent, the size of the inversible micelles was maintained at over 116 nm even with the solvent evaporation and re-hydration in PBS. Even strong detergents, such as sodium-dodecyl sulfate (SDS), failed to break down the micelles, as MA4-PEG-CA8 and CBA4-PEG-CA8 were able to form stable intermicellar crosslinkages in the presence of a nonpolar solvent. In contrast, in polar solvents, MA4-PEG-CA8 and CBA4-PEG-CA8 were not able to form core-inversible micelles and the final nanoparticles showed a smaller size as compared to other control micelles. Such smaller micelles could be easily destroyed in the presence of SDS (FIG. 1G), which was likely due to the lack of formation of enough boronate cross-linkages to stabilize the nanoparticles.

### Example 3. Drug Delivery

Loading hydrophobic and hydrophilic agents by STICK-NPs. Hydrophobic and hydrophilic agents (Table 1) were loaded into STICK-NPs by the solvent evaporation and cross-linked packaging method as described. Briefly, hydrophilic agents, MA4-PEG-CA8 (9 mg) and CBA4-PEG-CA8 (1 mg) were dissolved in 2 mL ultrapure water, followed by 3 min of sonication and the water was evaporated under vacuum to form a thin film in a round-bottom flask. Then the thin film was dispersed in 3 mL anhydrous chloroform with hydrophobic agents. The chloroform was evaporated under vacuum to form a thin film again. PBS buffer (1 mL) was added to re-hydrate the thin film, followed by 5 min of sonication. The unloaded free agents were removed by running the nanoparticle solutions through centrifugal filter devices (MWCO: 3 kDa, Microcon®). The hydrophobic and hydrophilic agents loaded STICK-NPs on the filters were recovered with PBS. The drug loading rate was calculated according to the calibration curve and concentrations of drug standard by the absorption intensity (such as Cy7.5), HPLC (such as vincristine) or inductively coupled plasma mass spectrometry (ICP-MS) (such as Gd-DTPA). The loading efficiency is defined as the ratio of agents loaded into nanoparticles to the initial agent content.

Drug release profile. STICK-NP@Cy@Gd was prepared to evaluate the in vitro release profile using dialysis cassettes (Pierce Chemical Inc.) with a 3 kDa MWCO. To make an ideal sink condition, 10 g charcoal was added in the release medium. The cassettes were dialyzed against PBS (pH7.4) at room temperature. The PBS at pH 7.4 was replaced with fresh PBS at pH 6.5 at 4 h. The concentration of CY7.5 and Gd-DTPA remaining in the dialysis cassette at various time points was measured by UV-vis spectroscopy and ICP-MS.

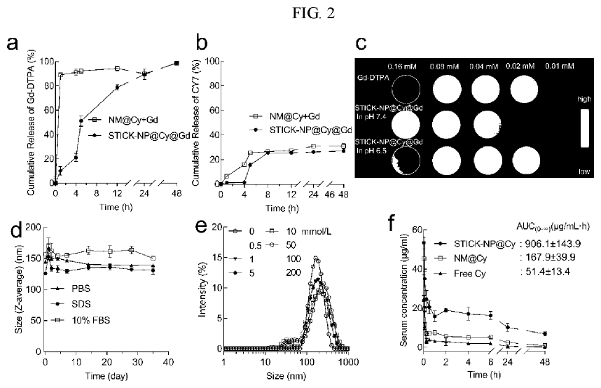

The distinctive drug loading in different compartments of STICK-NPs led to different drug release profiles of the hydrophilic and hydrophobic payloads in response to pH changes. Hydrophilic Gd-DTPA and hydrophobic Cy7.5 dye were used as model drugs for co-loading into STICK-NPs, and a drug release study was performed in pH 7.4 medium initially and then in pH 6.5 medium after 4 hours (FIG. 2A-2B). This experimental was purposely designed to model the two-stage in vivo drug release (pH 7.4 in blood and pH 6.5 in tumor microenvironment). Hydrophilic Gd-DTPA could not be loaded into NMs efficiently, and thus NM+ free Gd-DTPA was used in this study. FIG. 2A showed that free Gd-DTPA was released immediately, while Gd-DTPA was released from STICK-NPs at a much lower rate but could be accelerated upon changing to pH 6.5 solution. This was because hydrophilic Gd-DTPA was trapped between micelles and could gradually diffuse but only rapidly release upon pH-dependent cleavage of intermicellar crosslinkages. The release rate of hydrophobic Cy7.5 loaded in the hydrophobic interior of secondary micelles of STICK-NPs was dramatically slower than that of Gd-DTPA at pH 7.4, which is likely due to the hydrophobic property of Cy7.5 (FIG. 2B). At acidic pH, the release of Cy7.5 from STICK-NPs was slightly enhanced, probably due to the mild crosslinkage formed within the secondary micelles. In contrast, Cy7.5 loaded non-crosslinked non-targeting micelles (NM@Cy) showed faster drug release under pH7.4 and had minimal response to pH changes as there were no pH-responsive crosslinkages (FIG. 2B). These results demonstrated that STICK-NP can rapidly release hydrophilic drugs in a lower-pH responsive manner and deliver hydrophobic drugs into tumors through a secondary micelle release mechanism. Taking advantage of the co-loaded Cy7.5 and Gd-DTPA, STICK-NPs could potentially be applied for dual-modal imaging (magnetic resonance imaging (MRI) and near-infrared fluorescence (NIRF) imaging) (FIG. 2C; FIG. 8C-8E). Upon exposure to a lower pH environment, STICK-NP@Cy@Gd transformed and released hydrophilic Gd-DTPA, resulting in a recovered T1 signal comparable to that of free Gd-DTPA. The r1 of STICK-NP@Cy@Gd increased from 1.061 mM-1*s-1 to 4.447 mM-1*s-1 when the pH was changed from 7.4 to 6.5 (FIG. 8E).

The first biological barrier for brain tumor nanoparticle delivery is the strong destabilizing effects in blood circulation that includes: extreme dilution, an ionic environment, and interaction with blood proteins and lipoproteins (e.g. HDL, LDL), resulting in nanoparticle dissociation and premature drug release. Stabilized by inter-micellar crosslinkages, STICK-NP@Cy@Gd retained their size in PBS and even in the presence of 50 mM SDS and 10% FBS/PBS over a period of 35 days (FIG. 2D). Since STICK was dependent on the formation of the boronate ester bond between CBA and MA (glucose derivative with two cis-diols), there was a concern for the possible competition from the serum glucose resulting in the degradation of crosslinkages. Therefore, additional experiments were performed and demonstrated that crosslinkage was very stable at the physiological levels of glucose and up to a glucose concentration reaching 100 mmol/L (FIG. 2E). Of note, the level of serum glucose for normal human was around 3.9-5.5 mmol/L (70-100 mg/dL), and even patients suffering from diabetes are unlikely to reach a glucose level of 50 mmol/L. Additionally, STICK-NP performed exceptionally in a pharmacokinetic study in rats. Compared to conventional NM and free Cy7.5 formulations, STICK-NP@Cy@Gd increased the area under the curve (AUC(0-∞)) by 5.4 times and 17.6 times, respectively (FIG. 2F; Table 2). Besides, STICK-NP@Cy exhibited the highest Cmax (34.98±3.63 mg/L, or 5 times higher than NM@Cy), and longest t1/2z (34.66±12.13 hours, or 2 times longer than NM@Cy). These results strongly support that STICK-NPs exhibited superior stability during circulation and prevented premature drug release due to inter-micellar crosslinkages. Such improvements that significantly increase systemic circulation time offer a prolonged drug delivery window to brain tumors.

TABLE 2
Pharmacokinetic parameters for various formulations.

| Parameter | Unit | STICK-NP@Cy | NM@Cy | Free Cy |
|---|---|---|---|---|
| AUC_{(0-∞)} | μg/mL · h | 906.1 ± 143.9 | 167.9 ± 39.9 | 51.4 ± 13.4 |
| t_{1/2z} | h | 34.66 ± 12.13 | 17.14 ± 9.8 | 16.92 ± 0.78 |
| Cₘₐₓ | mg/L | 34.98 ± 3.63 | 7.91 ± 0.65 | 4.19 ± 0.4 |
| V_{z} | L/kg | 0.27 ± 0.051 | 0.7 ± 0.23 | 2.46 ± 0.51 |

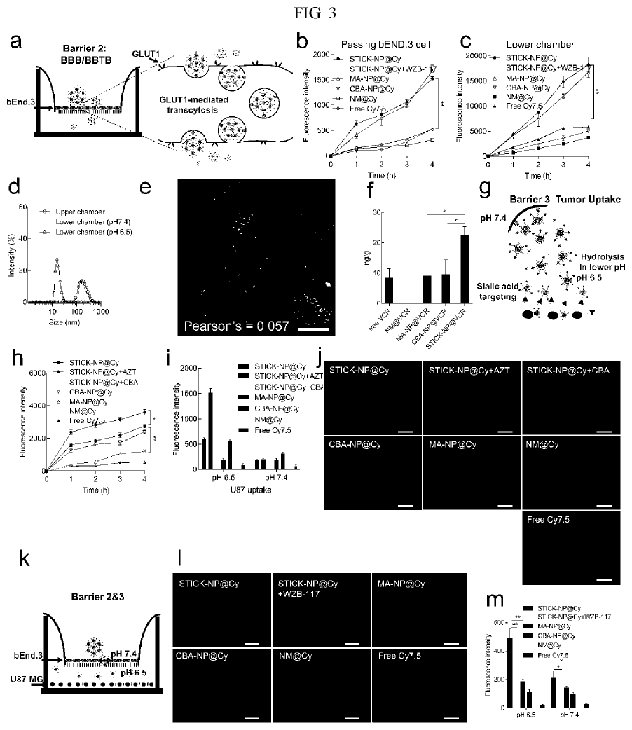

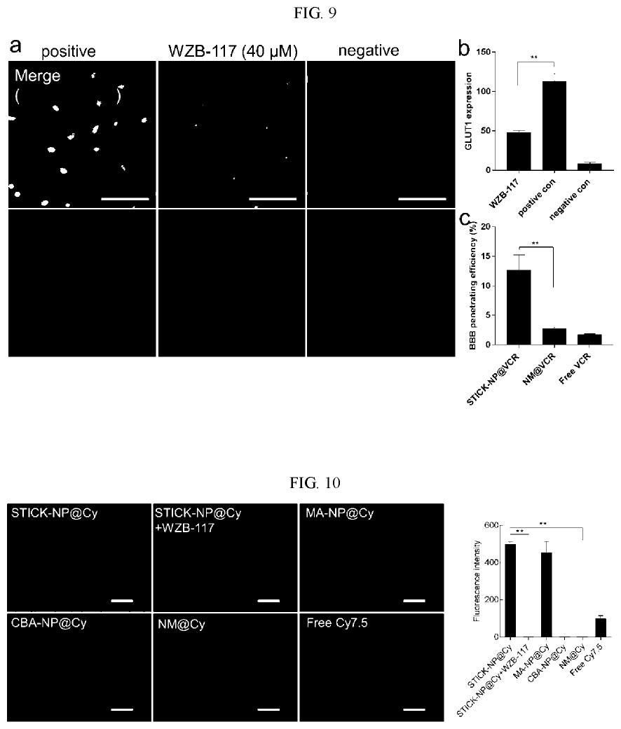

As orthotopic brain tumor model may not have intact BBB due to mechanical disruption, it was decided to validate the ability of the STICK-NPs for delivery of the poorly brain permeable chemotherapeutic drug, VCR for poorly brain permeable, in vitro and in normal Balb/c mice. Similarly, STICK-NP@VCR could transpass brain endothelial cells and deliver significantly higher VCR to the lower chamber, compared to free and NM@VCR in the BBB transwell modeling system (FIG. 9C). In the Balb/c model, at 6 hours post-injection, whole brains were harvested and tissue drug concentrations were measured by LC/MS. Around double amounts of VCR retained in the normal brain parenchyma after STICK-NP@VCR was demonstrated, compared to free VCR, or other non- or single targeting formulations (FIG. 3F). Collectively, these results confirmed that STICK-NPs could efficiently transverse the BBB/BBTB via GLUT1 mediated transcytosis.

Drug accumulation in brain tissue. 4-5 weeks-old female Balb/c mice (Envigo, Sacramento, Calif.) were i.v. injected with free VCR, NM@VCR, MA-NP@VCR, CBA-NP@VCR, and STICK-NP@VCR (n=4) at 2 mg/kg. Six hours later, animals were sacrificed and the whole brain was harvested immediately. Brain tissues were weighed and homogenized in PBS. VCR was extracted with methanol by 3 min sonication. Tissue VCR concentrations were determined by the validated LC-MS/MS methods.

In brief, the triple quadrupole LC-MS/MS system consisted of a 1200 series HPLC system (Agilent Technologies, USA) and a mass spectrometer (6420 triple Quad LC/MS, Agilent Technologies, USA). Chromatographic separation was achieved on a Waters XBridge-C18 (2.1 mm×50 mm, 3.5 μm) column at 40° C. with an isocratic mobile phase A was 10 mM ammonium acetate 0.10% formic acid aqueous and mobile phase B was acetonitrile.

The gradient was 0 min, 10% B; 0.8 min, 10% B; 2 min, 20% B; 3.0 min, 90% B; 3.5 min, 90% B; then back to 10% B in 0.5 min and equilibrated for 0.8 min for the next injection. The injection volume was 10 μL and the flow rate was 0.2 mL/min. VCR and vinblastine (as internal standard) were all ionized by ESI source in positive ion mode. The MS parameters were as follows: capillary, 5000 V; gas temperature, 320° C.; gas flow, 8 L/min; and nebulizer, 40 psi. Quantification was performed using multiple reaction monitoring (MRM) of the transition of m/z 825→765 with collision energy (CE) of 40 eV and fragmentor of 280 V for VCR, and m/z 811→355 with CE of 40 eV and fragmentor of 280 V for vinblastine. The system control and data analysis were performed by Mass Hunter Work station Software Qualitative Analysis (Version B.06.00) and Quantitative Analysis (Version B.05.02).

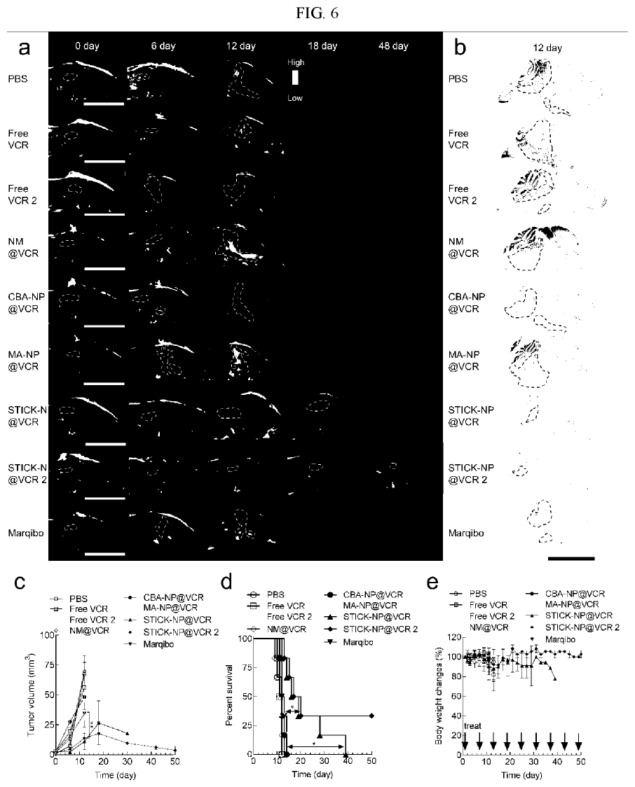

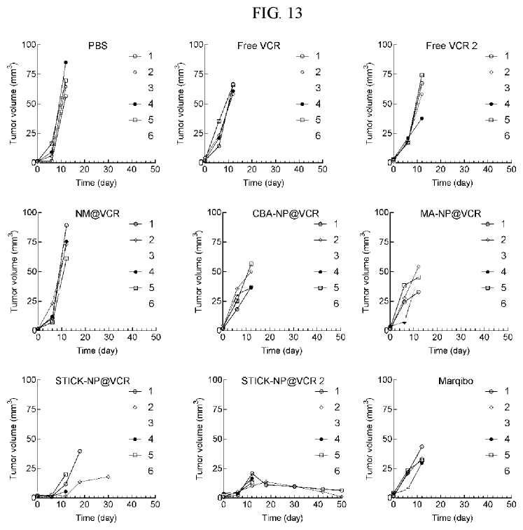

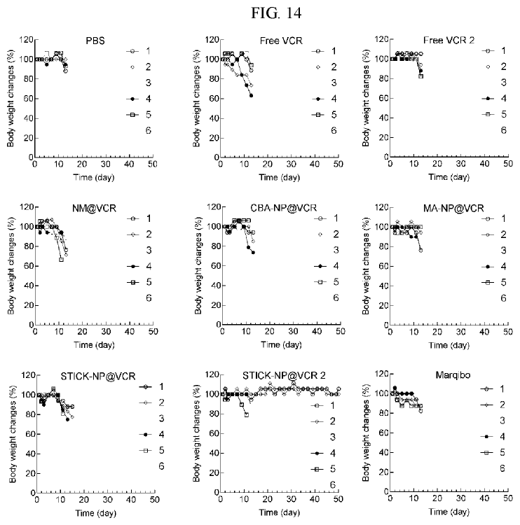

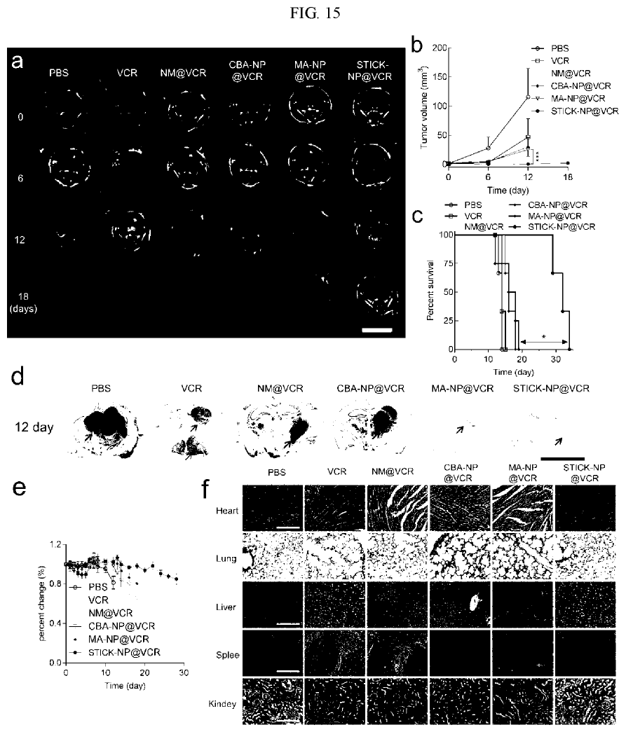

While VCR has well demonstrated anticancer activity, its effectiveness in brain tumors is limited due to its inability to penetrate the BBB/BBTB and dose-limiting neurotoxicity. Hence, STICK-NPs was employed to deliver VCR and evaluated their anti-cancer effects in a very aggressive and infiltrating orthotopic DIPG brain tumor model. Pediatric DIPG cells were injected into the pons of the SCID mouse brain to establish orthotopic model. After confirming the establishment of the DIPG brain tumors in mice using Gd-enhanced T1 weighted MRI (FIG. 6A), mice were randomly assigned into 9 groups: PBS, 1.5 mg/kg free VCR, NM@VCR, MA-NP@VCR, CBA-NP@VCR, STICK-NP@VCR and Marqibo (liposomal VCR), and two high dose groups, free VCR2 and STICK-NP@VCR2 (VCR 2 mg/mL) (n=6). Since this is a very aggressive DIPG model, free VCR (1.5 and 2 mg/kg), NM@VCR, MA-NP@VCR, CBA-NP@VCR, and Marqibo, all had minimal inhibition effects on tumor growth and failed to extend the survival of the animals compared to PBS control (FIG. 6A-6D). Very encouragingly, STICK-NP@VCR exhibited promising effects in hindering tumor growth (FIG. 6A-6C; FIG. 13) and almost doubled the survival times (21.3 days) compared to Marqibo, CBA-NP@VCR and MA-NP@VCR (survival time 12.5 days, 12 days and 12 days, respectively) (FIG. 6D). Even at the higher dose (2 mg/kg), VCR had no benefit in the survival time of DIPG bearing mice (FIG. 6A-6C). In contrast, STICK-NP@VCR at the equivalent dose level could further prolong the overall survival time, and 2 out of 6 mice in this group survived over 50 days. To achieve the best results, the remaining animals were continuously treated with 2 mg/kg of STICK-NP@VCR every 6 days. The orthotopic DIPG tumors in these mice were completely eradicated. During the treatment period, there were no significant changes in body weight, until the development of the neurological syndrome due to increased tumor burden and invasion (FIG. 6E; FIG. 14). Additionally, a similar efficacy study was performed in a more vascularized GBM orthotopic model in nude mice (FIG. 15). STICK-NP@VCR consistently outperformed other formulations with only a single dose of 2 mg/kg VCR. STICK@VCR significantly impeded tumor progression based on both MRI and histopathology (FIG. 15A, 15D) and prolonged the median survival times (34 days), compared to other formulations (all less than 17 days). Major organs were also harvested on day 12 post-treatment, and no major pathological changes were identified in all groups (FIG. 15F). STICK-NPs could efficiently deliver a high dose of the chemotherapeutic drug to the tumor site and eradicate brain tumors with limited toxicity. The disappointing anti-cancer results by either CBA or MA single targeting nanoparticles restates the need to consider the complexity and dynamic circumstances during brain tumor delivery.

### Example 4. Treating Diseases

Cell culture. The mouse bEnd.3 cells and human U87-MG cells were obtained from ATCC and were maintained in the DMEM, containing 10% fetal bovine serum (FBS) and 1% penicillin/streptomycin at 37° C. under 5% CO2 environment. U87-MG cells were transfected with GFP for imaging studies.

Transwell® culture system. To model the BBB/BBTB, Transwell® culture system was employed to culture bEnd.3 cells on the upper chamber with or without U87-MG cultured in the lower chamber. The pore size of Transwell was 0.4 μm and each well was seeded with 5×104 bEnd.3 cells. The integrity of bEnd.3 monolayer in vitro was evaluated by transendothelial electrical resistance. After 7 days, the transendothelial electrical resistance value reached over 200 Ω·cm² and was considered as the formation of tight junctions. U-87-MG cells were then cultured in the lower chamber overnight. STICK-NP@Cy (0.2 mg/mL Cy7.5) and other controls as indicated were placed in the upper chamber for 2 h allowing spontaneous transcytosis. The samples in the lower chamber were collected at different time points to detect Cy fluorescence and particle size (PBS was used instead of FBS) using DLS. Transwell was removed, and the pH values of the lower chamber medium were adjusted to pH 6.5 by 10 mM HCl or left at pH 7.4. Nanoparticle-containing medium was further left in the lower chamber with U87-MG cells for another hour allowing cell uptake. The U87-MG cell uptake in the lower chamber was monitored with a fluorescence microscope (BZ-X700, Keyence, Japan). Imaging was quantified and analyzed by Image J.

In vitro and in vivo penetration study. The second barrier encountered by the STICK-NPs is the BBB/BBTB, tight junctions formed by the brain microvessel endothelial cells. Excess ratios of MA (glucose derivative) on STICK-NPs are the first exposed targeting moiety for GLUT1 mediated endothelial cell transcytosis, while CBA is shielded in the STICK (FIG. 1A). Mouse brain endothelial cells (bEnd.3) cells were cultured in the top chamber of a Transwell® system and the formation of the tight junctions was confirmed by the transendothelial electrical resistance (TEER)>200 Ω·cm² (FIG. 3A). The evaluation of the total fluorescence intensities in the bEnd.3 cells (during transcytosis) (FIG. 3B; FIG. 10) and the medium in the lower chamber (post-transcytosis) (FIG. 3C) were performed at different time points after loading nanoparticles on the top chamber. FIG. 3B demonstrated that STICK-NP@Cy and MA-NP@Cy (also targeting GLUT1 via MA) had the highest intracellular signals among all groups. Consistent with this finding, STICK-NP@Cy and MA-NP@Cy groups had the highest tight-junction transversed amounts into the lower chambers (FIG. 3C). When GLUT1 was blocked by the GLUT1 inhibitor (WZB-117) (FIG. 9A-9B), the transverse of STICK-NP@Cy was diminished. The most intriguing finding was that the size of the STICK-NP@Cy remained similar before transcytosis (˜164 nm) and after transcytosis (˜146 nm) through bEnd.3 cells when comparing the size of STICK-NP@Cy in the upper and lower chambers (FIG. 3D). When the subcellular distribution of STICK-NP@DiD in bEnd.3 cells was assessed, it was discovered that STICK-NP@DiD did not co-localize with lysosome with a low Pearson's coefficient index of 0.057. Presumably, the low lysosomal pH (5.5) should have destroyed the crosslinkages and initiated the release of secondary smaller micelles if a lysosomal-dependent pathway occurred. Those collective evidenced support the notion that STICK-NP transpass BBB probably via a transytosis pathway and further detailed mechanism studies are undergoing.

The U87-MG three-dimensional spheroids were cultured according to the reported method. Briefly, U87-MG-GFP cells were seeded in U shaped bottom plate at the density of 1×104 cells/well. Four days later, the cells grew into tight spheroids with the diameter up to 400 μm. Tumor spheroids were then incubated with STICK-NP@DiD (under pH 7.4/6.5) or other controls for 24 h. Imaging was acquired by Leica confocal laser scanning microscopy to evaluate the degree of the nanoparticle penetration toward the center of the tumor spheroids. The imaging was further analyzed by Image J.

Orthotopic brain tumor models were established by injecting 2.5×104 DIPG (PDX) cells into the left side of brainstem of the female SCID mouse. The mice were injected with STICK-NP@DiD and NM@DiD (2.5 mg/kg for DiD). After 24 h, the mouse were sacrificed and injected with FITC-Dextran (70 K) to mark the blood vessel at 2 min before the sacrificing.

Cellular uptake assay. Lastly, after passing through the BBB, STICK-NPs then enter the acidic tumor microenvironment (Barrier 3). In response to the lower extracellular pH, STICK was broken down resulting in the release of secondary small micelles (FIG. 3D, 3G). CBA was originally shielded as part of STICK and now to be exposed after cleavage of crosslinking as the secondary tumor targeting moiety for brain tumors (FIG. TA and FIG. 3G). Next, the brain tumor cell targeting and cellular uptake abilities of secondary STICK-NPs using fluorescence imaging was investigated. Human U87-MG GBM cells were treated with STICK-NP@Cy and other control formulations under both pH 7.4 and pH 6.5 for 4 hours (FIGS. 3H-3I). Results demonstrated that the overall cellular uptake was relatively lower at pH 7.4 in all groups, including STICK-NPs with shielded CBA. In contrast, pretreatment with pH 6.5 exposed CBA which significantly enhanced brain tumor cell uptake of STICK-NP@Cy. Conversely, there was no significant enhancement in free Cy7.5, MA-NP@Cy, CBA-NP@Cy, and NM@Cy groups even with pre-treatment at pH 6.5. To further explore the potential role of sialic acid expression in the nanoparticle uptake, cells were treated with 3-Azidothymidine (AZT) to increase surface sialic acid expression. Such treatment further facilitated tumor cell uptake of STICK-NPs (pH 6.5) (FIG. 3H-3J). Furthermore, the CBA mediated cellular uptake of STICK-NPs (pH 6.5) could be radically blocked by excess free CBA (FIG. 3H-3J). These results proved that STICK-NPs could be effectively uptaken by brain tumor cells after transformation, which is likely due to the newly revealed CBA to enhance the sialic acid-mediated transcytosis. It was worth considering that under pH 6.5, CBA has a much higher affinity toward sialic acid than glucose (as MA), and thus would preferably bind to sialic acid on tumor cells.

To study the cellular uptake of STICK-NP@Cy, the bEnd.3 cells or U87-MG cells were seeded on 8-well chamber slides (10000 cells/well) and treated with STICK-NP@Cy and other controls (0.1 mg/mL Cy7.5) for 1 hour and washed by PBS three times. Cells were then fixed and stained with DAPI. Cell imaging was acquired using a Keyence fluorescence microscope. For quantitative study, bEnd.3 cells or U87-MG cells (10000 cells/well) were seeded in 96 well plate for overnight and then treated the cells with STICK-NP@Cy and other controls (0.1 mg/mL Cy7.5). Cells were harvested at 0 h, 1 h, 2 h, 3 h, and 4 h and washed with PBS. Total cells were lysed with 100 μL DMSO and the fluorescence intensity was measured by fluorescence spectrophotometer (RF-6000, Shimazu, Japan). To inhibit GLUT1 activity, bEnd.3 cells were pretreated with 40 μM of WZB-117 for 24 h before incubation with STICK-NP@Cy. For the tumor uptake study, U87-MG cells were pretreated with 40 μM of AZT for 24 h to alter the expression of surface sialic acid. To block the interaction, U87-MG cells were pre-incubated with excess free CBA (80 μM) for 24 h to compete for the binding sites with STICK-NP@Cy (pH 6.5) through the CBA targeting moiety in the secondary smaller micelles.

To model the combination of barrier 2 (BBB/BBTB) and barrier 3 (brain tumor uptake) in delivery to brain tumors, bEnd.3 cells were cultured in the upper chamber of Transwell and U87-MG brain tumor cells in the lower chamber (FIG. 3K). STICK-NP@Cy and other control NPs were loaded in the upper chamber for 1 hour and the pH of the medium in the lower chamber was adjusted to 7.4 or 6.5 for an additional 1 hour allowing U87-MG tumor cell uptake. As expected, FIG. 3L, m shows that STICK-NP@Cy (pH 6.5) group achieved the highest uptake in U87-MG cell compared to STICK-NP@Cy (pH7.4), MA-NP@Cy, CBA-NP@Cy, and NM@Cy (pH7.4 and 6.5) groups or free dye in the lower chamber. GLUT1 inhibition also impeded the final U87-MG cell uptake potentially due to decreased transcytosis (FIG. 3B-3C). Those results altogether provided a step-by-step validation of the mechanisms for the significantly enhanced drug delivery of STICK-NPs including BBB/BBTB transcytosis, transformation, and tumor cellular uptake. Importantly, single targeting nanoparticles either with CBA or MA may slightly improve the delivery to brain tumors but the efficiency was still sub-optimal in comparison.

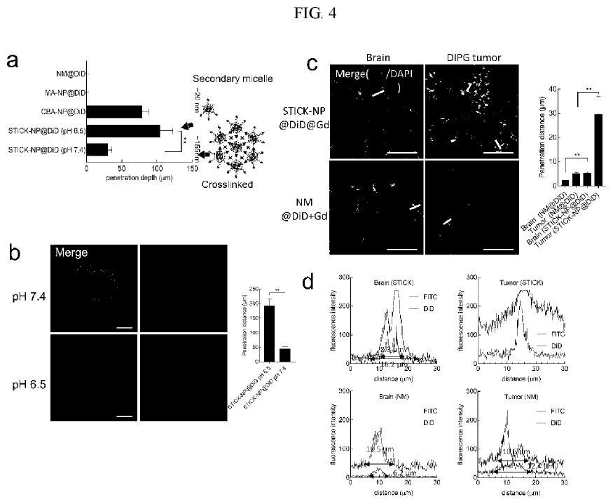

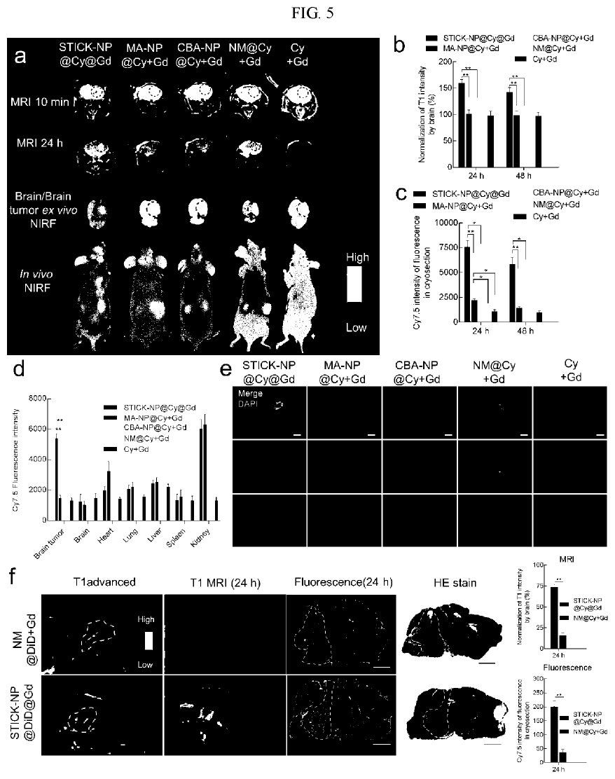

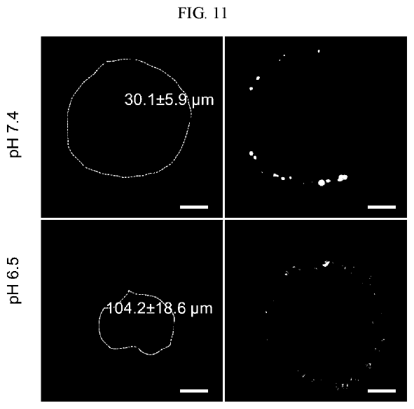

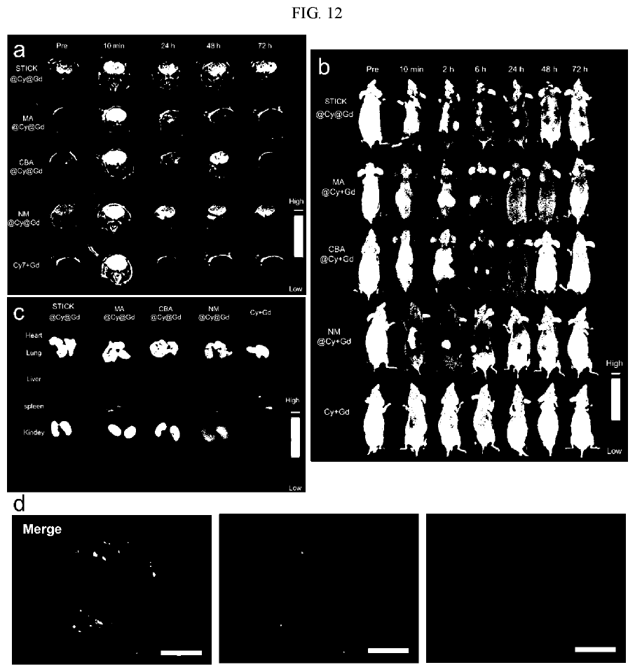

After transcytosis and transformation, STICK-NPs released numerous secondary micelles with a diameter of around 20 nm, which is more suitable for deep tissue penetration in tumors (FIGS. 1B, 1D). The three-dimensional multicellular spheroid system most resembles in vivo conditions and forms a compact extracellular matrix environment allowing for testing of drug penetration in vitro. To assess the size-dependent tissue penetration effects, the U87-MG neurosphere (˜400 μm) were incubated with STICK-NP@DiD and other control formulations under pH 7.4 or 6.5. After 24 hours, confocal fluorescence imaging of U87-MG spheroid showed that non-transformed STICK-NP@DiD (pH 7.4) group had poor penetration and lower penetration depth (30.1 μm±5.9 μm) (FIG. 4A; FIG. 11) due to its relatively large size (˜142 nm) (FIG. 1B). Upon pH-dependent transformation, STICK-NP@DiD (pH 6.5) possessed significantly superior penetration ability compared to STICK-NP@DiD (pH 7.4) and reached a similar depth compared to other nanoformulations with a small size (˜20 nm) (FIG. 4A; FIG. 1B; FIG. 11). Similar pH dependent transformation/penetration effects were further confirmed in the DIPG patient-derived xenograft (PDX) neurosphere (˜300 μm in diameter) (FIG. 4B). The pH-responsive feature actually equips STICK-NP with tumor selectivity. Accordingly, an orthotopic DIPG model was employed to evaluate the degree of the tissue penetration of STICK-NPs at both normal brain and acidic tumor sites. FIGS. 4C-4D showed that at 24 hours, STICK-NP@DiD were able to penetrate into DIPG tumor tissue around 30 μm far from the blood vessels. In contrast, in the normal brain parenchyma (reported dog brain parenchyma pH was 7.13), STICK-NP@DiD only penetrated around 5 μm beyond the blood vessel. Meanwhile, NM@DiD control had minimal normal brain penetration ability (FIG. 4C). Along with the in vitro studies, it was concluded that STICK-NP could be selectively responsive to the acidic environment to release secondary nanoparticles with newly revealed CBA targeting moiety allowing better tumor tissue penetration and tumor cell uptake. With the pH selectivity, STICK-NP would have limited normal tissue penetration and less concern for neurotoxicity.

Anti-cancer efficacy study in orthotopic brain tumor models. Orthotopic brain tumor models were established by injecting 2.5×104 DIPG (PDX) cells into the left side of brainstem of the female SCID mouse or 5×104 GBM (U87-MG) cells into the left side of brain of the female nude mouse brain as described above. After confirming the establishment of brain tumors, mice were randomly assigned into different groups. Tumor size was monitored using advanced T1-weight imaging (TR/TE=300 ms/15 ms). For imaging study, mice were injected with 250 mg/kg Gd-DTPA. Tumor size of DIPG model was calculated from the aggregation of tumor area in different MRI slices, 1 mm thick. Tumor size of GBM model was calculated as the followed equation:

$$Tumor volume ( {mm}^{3} ) = \frac{L \times {W}^{2}}{2}$$

where W is the width of the tumor and the L is the length of the tumor (W<L). One mouse per group was sacrificed on day 12 after MRI imaging, and organs and brain with tumors were harvested for histopathology evaluation. Animals were continuously monitored their appearance, behavior, and body weight. Once the body weight loss>20%, animals were considered as reaching the humane endpoint.

The targeted delivery of STICK-NPs was further investigated in an orthotopic DIPG PDX model. Gd-enhanced T1-weighted MRI was first used to locate DIPG. After the clearance of the Gd signal, the mice were re-injected with DiD+Gd, NM@DiD+Gd, and STICK-NPs@Gd@DiD and re-imaged at 16 hours post-injection (FIG. 5F). As shown in FIG. 5F, STICK-NPs@Gd@DiD selectively and efficiently concentrated at the tumor sites as shown in both imaging modalities. The imaging studies served as strong support that STICK-NP@Cy@Gd could specifically deliver payloads to the tumor sites allowing accurate imaging-guided drug delivery and potential utilization for delineation of tumor margins during surgery. In contrast, single target formulations, MA-NPs, and CBA-NPs which previously showed their targeting effects in vitro, were not able to deliver sufficient payload to orthotopic brain tumors in vivo.

### Example 5. Imaging

ARS based fluorescence assay. ARS is a catechol dye displaying dramatic changes in absorption and fluorescence intensity upon binding to boronic acid. ARS based fluorescence assay was utilized to confirm the formation of boronate ester bonds in solution. Briefly, ARS (0.1 mg/mL) was mixed with the CBA4-PEG-CA8 (2.5 μM) and different concentrations of MA4-PEG-CA8 (0˜ 40 μM). The change of fluorescence intensity (em: 585 nm, ex: 468 nm) of ARS was measured with a fluorescence spectrophotometer (Shimadzu, RF-6000).

Establishment of orthotopic brain model and studies for optical and MR imaging. An orthotopic PDX GBM model was next utilized to evaluate the biodistribution of STICK-NPs@Cy@Gd using the dual-modality imaging: NIRF imaging (Cy7.5) and MRI (Gd-DTPA) (FIG. 5A). At 10 min post-injection, all groups had increased overall brain MRI T1 weighted signals (FIG. 5A). At 24 and 48 hours post-injection, STICK-NP@Cy@Gd groups had both significantly higher T1-weighted MRI signal intensity (FIGS. 5A-5B) and Cy7.5 fluorescence intensity (FIGS. 5A, 5C, 5D) at the tumor sites, compared to free Cy7.5+Gd, NM@Cy+Gd, CBA-NP@Cy+Gd, and MA-NP@Cy+Gd groups. It is important to note that unlike in STICK-NPs, hydrophilic Gd-DTPA could not be loaded in the NM, CBA-NPs, and MA-NPs and thus were injected as free Gd-DTPA in those groups along with Cy7.5 loaded nanoparticles as control groups. The NIRF or T1-weight MRI signals of STICK-NP@Cy@Gd were maintained in the tumors for the longest time and only returned to baseline at 72 hours post-injection (FIG. 12A). Although only used ⅓ of the clinical dose of Gd-DTPA was used, it appeared that this particular PDX model exhibited poor permeability, evidenced by the minimal T1 signals of Gd-DTPA presented at the tumors sites at 10 min (FIG. 5A). Nevertheless, STICK-NPs could still efficiently target, penetrate, and retain in the PDX GBM model.

To further dissect the target delivery efficiency and selectivity into the brain tumor, another set of mice were sacrificed at 24 hours post nanoparticle administration and major organs/brain with brain tumors were harvested for ex vivo NIRF imaging. Biodistribution was assessed based on the Cy7.5 signals in the brain and other major organs. As shown in FIGS. 5A, 5D, 12B, and 12C, STICK-NPs could specifically deliver a higher concentration of Cy7.5 to the orthotopic PDX GBM tumors compared to other major organs, excepting the kidney, which could potentially be the clearance route for Cy7.5 dye. The STICK-NPs treated group had a significantly higher accumulation of the Cy7.5 signals at the brain tumor sites, comparing to free Cy7.5+Gd and NM@Cy+Gd. NIRF imaging of cryosections from the orthotopic brain tumors in the STICK-NPs group exhibited a strong correlation between tumor cells (green) and Cy7.5 (red) (FIG. 5E; FIG. 12D) with a calculated Pearson's coefficient index of up to 0.637. Meanwhile, the normal brain had minimal uptake suggesting the excellent tumor selectivity of STICK-NPs (FIGS. 5C,5E). The semi-quantitative imaging analysis demonstrated that orthotopic glioblastoma PDX tumor had around 1.5 times and 4 times higher signals than adjacent normal brain tissues on MRI and NIRF imaging, respectively (FIGS. 5B, 5D).

Patient derived-xenograft (PDX) glioblastoma was kindly provided by Dr. C. David James from UCSF. Cells were previously transfected with GFP. To establish an orthotropic brain tumor, 5 μl of PDX cells (1×107/mL) or U87 (1×107/mL) were injected into the right striatum area of the mouse with the aid of a mouse stereotactic instrument (Stoelting). Cells were injected within 5 min and mice were allowed to rest another 5 min under general anesthesia. Animals received post-surgery for pain management for 3 days. Two weeks later, animals were intravenously administrated with STICK-NP@Cy@Gd and other control groups as indicated (Cy7.5: 10 mg/kg; Gd: 25 mg/kg). The in vivo near infrared red fluorescence imaging was acquired at different time points as indicated using Kodak imaging station (4000 MM). The same mice were also subjected to T1 weighted MR imaging for the brain at 0 min, 10 min, 24 h, 48 h, and 72 h. Bruker Biospec 7T MRI scanner was used to record imaging through the coronal cross-sectional view. The following parameters were used for all T1 weighted MR images recorded: TR=400 ms; TE=15 ms; matrix=256×256; FOV=20×20 mm2. After 24 h post imaging, mice were sacrificed, and all organs were harvested including tumor containing brain for ex vivo imaging. The whole brain with the tumor was fixed in the optimum cutting temperature compound. 10 μm of cryo-section was used for fluorescence microscopy imaging (Keyence), while the nuclei were stained with DAPI.

In summary, the STICK technology provides a simple but smart solution in tackling multiple barriers in drug delivery to brain tumors. STICK was designed based on a unique pair of two targeting moieties which could also form a stimuli-responsive bond, such as glucose derivatives and boronic acid families which could form pH-responsive boronate crosslinkages. In the current STICK approach, the targeting moieties (CBA or MA) serve much more than targeting purpose. They are integrated into the nanoparticle architecture and significantly contribute the desirable characteristics (e.g. stability, stimuli-responsiveness, transformability and versatile drug loading capability) and overall delivery performance of these nanoparticles. Such a unique STICK design clearly distinguished itself from previously published dual targeting systems. STICK strategy is introduced into well-characterized micelle formulation and showed that STICK-NPs could survive in the bloodstream and sequentially STICK into the BBB/BBTB and brain tumor cells, respectively. STICK-NPs were demonstrated to overcome the destabilizing environment in blood with the inter-micellar crosslinkages formed by MA (exposed) and CBA (shielded) and showed significantly prolonged circulation time allowing a wider brain tumor targeting window (FIG. 1). During circulation, surface excess MA on the nanoparticle could facilitate GLUT1-mediated transcytosis through BBB/BBTB to “actively” target brain tumors (FIG. 3). Subsequently, the STICK was cleaved after encountering the intrinsic acidic pH at the tumor sites, triggering the transformation into secondary smaller nanoparticles for deep tumor tissue penetration (FIG. 4), and revealing the secondary targeting moiety, CBA against the sialic acid overexpressed in tumor cells for enhanced cellular uptake (FIG. 5). The pH-dependent selectivity further endowed their biosafety features. In the orthotopic glioblastoma and DIPG mouse models, STICK-NPs effectively delivered both hydrophobic and hydrophilic image agents to tumor sites for the dual-modality imaging. Most excitingly, STICK-NP@VCR exhibited superior brain tumor inhibition effect and dramatically prolonged survival time even in the most aggressive and VCR-resistant DIPG model in comparison to the single targeting formulations (FIG. 6). These promising results highlighted the unique feature of STICK at overcoming different complicated barriers and the importance of considering all the obstacles during nanoparticle design for successful brain tumor delivery. Given the versatile drug loading capability, STICK-NP could provide the immediate second hope to deliver the most advanced epigenetic modulating agents, such as HDAC and EZH2 inhibitors, which efficacies were greatly hindered by the BBB/BBTB resulting in failed clinical trials. The STICK strategy provides noteworthy opportunities to apply the approach to many other nanoformulation designs against dynamic and entanglement biological barriers and also have an impact in advancing the drug development/delivery for aggressive brain tumors.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula I:
(R′)ₘ-D¹-L¹-PEG-L²-D²-(R²)ₙ (I)
wherein:
each R¹ is independently a peptide, 1,2-dihydroxy compound, or boronic acid derivative;
each R² is independently cholic acid or a cholic acid derivative;
D¹ and D² are each independently a dendritic polymer having a single focal point group, and a plurality of branched monomer units X;
each branched monomer unit X is a diamino carboxylic acid, a dihydroxy carboxylic acid or a hydroxyl amino carboxylic acid;
L¹ and L² are each independently a bond or a linker linked to the focal point group of the dendritic polymer;
PEG is a polyethylene glycol (PEG) polymer having a molecular weight of 1-100 kDa;
subscript m is an integer from 2 to 8; and
subscript n is an integer from 2 to 16.

2. The compound of claim 1, wherein each R¹ is independently a peptide, 1,2-dihydroxy compound, sugar compound, glucose, or glucose derivative.

3. The compound of claim 1 or 2, wherein each R¹ is independently angiopep-2, levodopa, cellulose, oligosaccharide, cyclodextrin, maltobionic acid, glucosamine, sucrose, trehalose, or cellobiose.

4. The compound of any one of claims 1 to 3, wherein each R¹ is independently maltobionic acid.

5. The compound of claim 1, wherein each R¹ is independently a boronic acid derivative.

6. The compound of claim 1 or 5, wherein each R¹ is independently a 3-carboxy-5-nitrophenylboronic acid, 4-carboxyphenylboronic acid, 3-carboxyphenylboronic acid, 2-carboxyphenylboronic acid, 4-(hydroxymethyl)phenylboronic acid, 5-bromo-3-carboxyphenylboronic acid, 2-chloro-4-carboxyphenylboronic acid, 2-chloro-5-carboxyphenylboronic acid, 2-methoxy-5-carboxyphenylboronic acid, 2-carboxy-5-pyridineboronic acid, 6-carboxy-2-fluoropyridine-3-boronic acid, 5-carboxy-2-fluoropyridine-3-boronic acid, 4-carboxy-3-fluorophenylboronic acid, or 4-(bromomethyl)phenylboronic acid.

7. The compound of any one of claims 1, 5, or 6, wherein each R¹ is independently 4-carboxyphenylboronic acid.

8. The compound of any one of claims 1 to 7, wherein each R² is independently cholic acid, (3α,5β,7α,12α)-7,12-dihydroxy-3-(2,3-dihydroxy-1-propoxy)-cholic acid (CA-4OH), (3α,5β,7α,12α)-7-hydroxy-3,12-di(2,3-dihydroxy-1-propoxy)-cholic acid (CA-5OH), or (3α,5β,7α,12α)-7,12-dihydroxy-3-(3-amino-2-hydroxy-1-propoxy)-cholic acid (CA-3OH-NH₂).

9. The compound of any one of claims 1 to 8, wherein each R² is cholic acid.

10. The compound of any one of claims 1 to 9, wherein each X is independently 2,3-diamino propanoic acid, 2,4-diaminobutanoic acid, 2,5-diaminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid.

11. The compound of any one of claims 1 to 10, wherein each X is lysine.

12. The compound of any one of claims 1 to 11, wherein L¹ is a bond.

13. The compound of any one of claims 1 to 12, wherein L² is a bond.

14. The compound of any one of claims 1 to 13, wherein PEG has a molecular weight of 1 to 20 kDa.

15. The compound of any one of claims 1 to 14, wherein PEG has a molecular weight of about 5 kDa.

16. The compound of any one of claims 1 to 15, wherein subscript m is 4 and subscript n is 8.

17. The compound of any one of claims 1 to 16, wherein the compound has the structure of Formula (Ia):

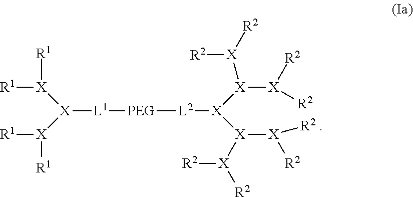

18. The compound of any one of claims 1 to 17, wherein the compound has the structure of Formula (Ib):

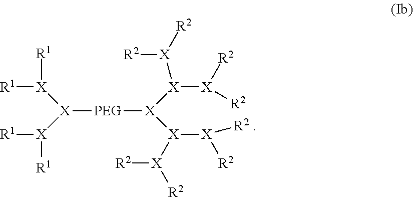

19. The compound of claim 18,
wherein:
each R¹ is maltobionic acid;
each R² is cholic acid;
each X is lysine; and
PEG has a molecular weight of about 5 kDa.

20. The compound of claim 18,
wherein:
each R¹ is 4-carboxyphenylboronic acid;
each R² is cholic acid;
each X is lysine; and
PEG has a molecular weight of about 5 kDa.

21. A nanoparticle comprising a plurality of first and second conjugates, wherein:
each first conjugate is a compound of claim 2;
each second conjugate is a compound of claim 5; and
the plurality of conjugates self-assemble by forming crosslinking bonds to form a nanoparticle such that the interior of the nanoparticle comprises a hydrophilic interior comprising a plurality of micelles with a hydrophobic core.

22. A nanoparticle comprising a hydrophilic exterior and interior, wherein the nanoparticle interior comprises a hydrophilic interior comprising a plurality of micelles having a hydrophobic core and hydrophilic micelle exterior, wherein each micelle comprises a plurality of first and second conjugates, wherein:
each first conjugate is a compound of claim 2;
each second conjugate is a compound of claim 5; and
the plurality of first and second conjugates self-assemble by forming crosslinking bonds to form the micelle with the hydrophobic core, with the crosslinking bonds on the hydrophilic micelle exterior.

23. The nanoparticle of claim 21 or 22, wherein the first conjugate is a compound of claim 19, and the second conjugate is a compound of claim 20.

24. The nanoparticle of any one of claims 21 to 23, wherein the nanoparticle further comprises a hydrophilic drug or imaging agent.

25. The nanoparticle of claim 24, wherein the hydrophilic drug or imaging agent is gadopentetic acid (Gd-DTPA), indocyanine green (ICG), cisplatin, gemicitabine, doxorubicin hydrochloride (DOX-HCl), or cyclophosphamide.

26. The nanoparticle of any one of claims 21 to 25, wherein the nanoparticle further comprises a hydrophobic drug or imaging agent.

27. The nanoparticle of claim 26, wherein the hydrophobic drug or imaging agent is cyanine 7.5 (Cy7.5), 1,1′-Dioctadecyl-3,3,3′,3′-tetramethylindodicarbocyanine 4-chlorobenzenesulfonate (DiD), doxorubicin (DOX), vincristine (VCR), everolimus, carmustine, lomustine, temozolomide, lenvatinib mesylate, sorafenib tosylate, regorafenib, Irinotecan, paclitaxel (PTX), Docetaxel, BET inhibitors, OTX015, BET-d246, ABBV-075, I-BET151, I-BET 762, HDAC inhibitors, Valproic acid, Vorinostat, Panobinostat, Entinostat, Ricolinostat, AR-42, JMJD3 inhibitors, GSKJ4, EZH2 inhibitors, Tazemetostat, GSK2816126, MC3629, EGFR inhibitors, Gefitinib, erlotinib, Lapatinib, Osimertinib, AZD92291, IDH inhibitors, enasidenib, ivosidemib, Notch inhibitors, RO4929097, CDK4/6 inhibitors, Palbociclib, Ribociclib, Abemaciclib, PI3K/Akt/mTOR inhibitors, Rapamycin, Buparlisib, Curcumin, or Etoposide.

28. The nanoparticle of any one of claims 21 to 27, wherein the ratio of the first conjugate to the second conjugate is about 10:1, 9:1, 5:1, 1:1, 1:5, or 1:10.

29. The nanoparticle of any one of claims 21 to 28, wherein the ratio of the first conjugate to the second conjugate is about 9:1.

30. A method of delivering a drug, the method comprising:
administering a nanoparticle of any one of claims 21 to 29, wherein the nanoparticle further comprises a hydrophilic and/or hydrophobic drug and a plurality of cross-linked bonds; and
cleaving the cross-linked bonds in situ, such that the drug is released from the nanoparticle, thereby delivering the drug to a subject in need thereof.

31. The method of claim 30, wherein the hydrophilic and/or hydrophobic drug is doxorubicin hydrochloride (DOX-HCl), doxorubicin (DOX), vincristine (VCR), or paclitaxel (PTX).

32. A method of treating a disease, the method comprising administering a therapeutically effective amount of a nanoparticle of any one of claims 21 to 29, wherein the nanoparticle further comprises a hydrophilic and/or hydrophobic drug, to a subject in need thereof.

33. The method of claim 32, wherein the disease is cancer.

34. The method of claim 32 or 33, wherein the disease is glioblastoma, diffuse intrinsic pontine glioma, brain metastases, lung cancer, breast cancer, colon cancer, kidney, cancer, or melanoma.

35. The method of claim 32, wherein the hydrophilic and/or hydrophobic drug is doxorubicin hydrochloride (DOX-HCl), doxorubicin (DOX), vincristine (VCR), or paclitaxel (PTX).

36. A method of imaging, comprising:
administering an effective amount of a nanoparticle of any of claims 21 to 29, wherein the nanoparticle further comprises a hydrophilic and/or hydrophobic imaging agent to a subject in need thereof; and
imaging the subject.

37. The method of claim 36, wherein the hydrophilic and/or hydrophobic imaging agent is gadopentetic acid (Gd-DTPA), indocyanine green (ICG), cyanine 7.5 (Cy7.5), or 1,1′-Dioctadecyl-3,3,3′,3′-tetramethylindodicarbocyanine 4-chlorobenzenesulfonate (DiD).
